(19) United States
(12) Patent Application Publication
(10) Pub. No.: US 2026/0248395 A1
(43) Pub. Date: Aug. 27, 2026

(54) VIRTUAL ANGIOGRAM SYSTEMS AND METHODS FOR ASSESSMENT OF ANEURYSMS

(71) Applicants: GEORGE MASON UNIVERSITY, Fairfax, VA (US); THE REGENTS OF THE UNIVERSITY OF CALIFORNIA, Oakland, CA (US)

(72) Inventors: Juan Raul CEBRAL, Vienna, VA (US); Naoki KANEKO, Los Angeles, CA (US)

(21) Appl. No.: 19/547,231

(22) Filed: Feb. 23, 2026

Related U.S. Application Data
(60) Provisional application No. 63/763,028, filed on Feb. 25, 2025.

(51) Int. Cl. A61B 5/02; A61B 5/026; G06F 30/28

(52) U.S. Cl. CPC A61B 5/02014; A61B 5/0263; G06F 30/28

(57) ABSTRACT

A virtual angiogram can be performed by simulating transport of contrast agent through a model of a portion of a vasculature of a patient. The simulation can be based on a patient-specific pulsatile flow velocity field of the vasculature portion. At least one contrast retention metric for an aneurysm in the vasculature portion can be determined based on the virtual angiogram. The contrast retention metric can be indicative of flow stagnation in the aneurysm. In some aspects, the aneurysm can be identified as unstable based at least in part on the at least one contrast retention metric. Responsive to the identification, a treatment can be administered for the aneurysm even though it has not yet ruptured. Alternatively or additionally, the aneurysm can be identified as ruptured, for example, to discriminate between multiple potentially-ruptured aneurysms in the patient for treatment.

### CROSS-REFERENCE TO RELATED APPLICATION(S)

The present application claims the benefit of and priority under 35 U.S.C. § 119(e) to and is a non-provisional of U.S. Provisional Application No. 63/763,028, filed Feb. 25, 2025, and entitled “Contrast Retention in PCOM Aneurysms: A Possible Marker for Aneurysm Instability,” which is hereby incorporated by reference herein in its entirety.

### STATEMENT REGARDING FEDERALLY SPONSORED RESEARCH

This invention was made with government support under NS121286 and NS097457 awarded by the National Institutes of Health. The government has certain rights in the invention.

### FIELD

The present disclosure relates generally to analysis of and/or medical diagnosis related to aspects of a patient's vasculature, and more particularly, to systems and methods employing a virtual angiogram for assessment of one or more aneurysms, for example, identification of an aneurysm as unstable or ruptured.

### BACKGROUND

Aneurysms are abnormal focal dilations of a blood vessel that can develop throughout the vasculature of a patient. The clinical management of an unruptured aneurysm often requires balancing the risk of spontaneous rupture and progressive instability against the inherent risks associated with surgical or endovascular interventions. Consequently, accurate and timely assessment of aneurysms may support more efficient patient triage and more effective treatment planning. Conventional techniques for assessing aneurysm risk often rely on static morphological parameters, such as maximum diameter, volume, or aspect ratio of the aneurysm. However, these localized anatomical metrics often fail to adequately capture the complex hemodynamic conditions that drive aneurysm growth and wall degradation. While dynamic assessment techniques exist, they frequently require highly invasive catheterization procedures or otherwise struggle to accurately predict aneurysm instability or discriminate between ruptured and unruptured aneurysms.

Embodiments of the disclosed subject matter may address one or more of the above-noted problems and disadvantages, among other things.

### SUMMARY

Embodiments of the disclosed subject matter provide systems and methods for assessment of an aneurysm in a patient via a virtual angiogram. One or more contrast retention metrics can be calculated based on the virtual angiogram. In some embodiments, at least one of the contrast retention metrics can be compared to a predetermined threshold (or a predetermined range), where the aneurysm is identified as unstable when the associated metric above the threshold (or outside the range). Alternatively, in some embodiments, the contrast retention metrics can be used to identify at least one of a plurality of aneurysms as ruptured, for example, where a patient has presented with symptoms of a ruptured aneurysm, but it may otherwise be difficult or impossible to determine which aneurysm has ruptured via conventional imaging. In some embodiments, the use of the virtual angiogram can allow an aneurysm to be identified as either unstable or ruptured using less invasive imaging modalities, for example, without requiring a physical intra-arterial catheterization of the patient.

In one or more embodiments, a method can comprise performing a virtual angiogram by simulating transport of contrast agent through a model of a portion of a vasculature of a patient based on a patient-specific pulsatile flow velocity field of the vasculature portion. The vasculature portion can include an aneurysm that has not ruptured. The method can further comprise determining at least one contrast retention metric for the aneurysm based on the virtual angiogram, the at least one contrast retention metric being indicative of flow stagnation in the aneurysm. The method can also comprise identifying the aneurysm as unstable based at least in part on the at least one contrast retention metric. In some embodiments, the method can further comprise physically administering an endovascular, surgical, or radiosurgical treatment to stabilize, occlude, or obliterate the identified aneurysm.

In one or more embodiments, a method can comprise performing a virtual angiogram by simulating transport of contrast agent through a model of a portion of a vasculature of a patient based on a patient-specific pulsatile flow velocity field of the vasculature portion. The vasculature portion can include a plurality of aneurysms, and at least one of the aneurysms may be ruptured. The method can further comprise determining at least one contrast retention metric for each of the plurality of aneurysms based on the virtual angiogram. Each contrast retention metric can be indicative of flow stagnation in the respective aneurysm. The method can also comprise identifying at least one of the plurality of aneurysms as ruptured based at least in part on the contrast retention metrics. In some embodiments, the method can further comprise physically administering an endovascular, surgical, or radiosurgical treatment to stabilize, occlude, or obliterate the identified aneurysm.

In one or more embodiments, a system can comprise one or more processors and one or more non-transitory media storing computer-readable instructions that, when executed by the one or more processors, cause the one or more processors to perform a virtual angiogram by simulating transport of contrast agent through a model of a portion of a vasculature of a patient based on a patient-specific pulsatile flow velocity field of said vasculature portion, the vasculature portion including at least one aneurysm; determine at least one contrast retention metric for the at least one aneurysm based on the virtual angiogram, the at least one contrast retention metric being indicative of flow stagnation in the respective aneurysm; and identify the at least one aneurysm as stable, unstable, or ruptured based at least in part on the at least one contrast retention metric.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

Embodiments will hereinafter be described with reference to the accompanying drawings, which have not necessarily been drawn to scale. Where applicable, some elements may be simplified or otherwise not illustrated in order to assist in the illustration and description of underlying features. Throughout the figures, like reference numerals denote like elements.

FIGS. 1A-1B are process flow diagrams illustrating aspects of an aneurysm assessment method employing a virtual angiogram, according to one or more embodiments of the disclosed subject matter.

FIG. 1C illustrates an exemplary implementation of an aneurysm assessment method, according to one or more embodiments of the disclosed subject matter.

FIG. 1D is a graph of time density curves generated in the exemplary implementation of FIG. 1C, according to one or more embodiments of the disclosed subject matter.

FIG. 1E is a graph of regional metrics derived from the time density curves of FIG. 1D, according to one or more embodiments of the disclosed subject matter.

FIG. 2A is a simplified schematic illustrating operational aspects of components of an aneurysm assessment system, according to one or more embodiments of the disclosed subject matter.

FIG. 2B illustrates an exemplary user interface or display of an aneurysm assessment system, according to one or more embodiments of the disclosed subject matter.

FIG. 2C depicts a generalized example of a computing environment in which the disclosed technologies may be implemented.

FIG. 3A is a graph of a receiving operating curve (ROC) for unruptured aneurysms versus ruptured aneurysms, where the y-axis represents sensitivity for the true positive rate (TPR) and the x-axis represents 1-sensitivity for the false positive rate (FPR), and AUC represents the area under the ROC.

FIG. 3B is a graph of ROC for unruptured aneurysms having a size less than 7 mm versus ruptured aneurysms having a size less than 7 mm, where the y-axis represents sensitivity for TPR and the x-axis represents 1-sensitivity for FPR, and AUC represents the area under the ROC.

FIG. 3C is a graph of ROC for stable aneurysms versus unstable (growing) aneurysms, where the y-axis represents sensitivity for TPR and the x-axis represents 1-sensitivity for FPR, and AUC represents the area under the ROC.

FIG. 4 shows Spearman correlation coefficients between flow stagnation metrics (rows) and aneurysm geometric parameters (columns), as well as linear regression R² correlation coefficients and scatter plots for two metric-parameter pairs, where IT represents transit time, MRT represents mean residence time, TP represents time to peak, TW represents time to washout, SK represents skewness, Asize represents aneurysm size, Nsize represents neck size, Depth represents aneurysm depth, AR represents aspect ratio, BF represents bottleneck factor, BL represents bulge location, SizeR represents size ratio, VOR represents volume to ostium ratio, UI represents undulation index, EI represents ellipticity index, NSI represents non-sphericity index, CP represents conicity parameter, and MLN represents mean Gaussian curvature.

FIGS. 5A-5E are images of a vascular model for a stable aneurysm without contrast retention showing an inflow stream (FIG. 5A), flow pattern (FIG. 5B), vortex corelines (FIG. 5C), wall shear stress (FIG. 5D), and divergence (blue arrow 502) and swirling (orange arrow 504) critical points.

FIGS. 5F-5G show selected frames at respective times (t₁-t₈) and time density curves, respectively, generated by a virtual angiogram for the vascular model illustrated in FIGS. 5A-5E.

FIGS. 6A-6E are images of a vascular model for an unstable stable aneurysm with contrast retention showing an inflow stream (FIG. 6A), flow pattern (FIG. 6B), vortex corelines (FIG. 6C), wall shear stress (FIG. 6D), and divergence (blue arrow 602) critical point.

FIGS. 6F-6G show selected frames at respective times (t₁-t₈) and time density curves, respectively, generated by a virtual angiogram for the vascular model illustrated in FIGS. 6A-6E.

FIGS. 7A-7E are images of a vascular model for a ruptured aneurysm with contrast retention showing an inflow stream (FIG. 7A), flow pattern (FIG. 7B), vortex corelines (FIG. 7C), wall shear stress (FIG. 7D), and divergence (blue arrows 702) and swirling (orange arrow 704) critical points.

FIGS. 7F-7G show selected frames at respective times (t₁-t₈) and time density curves, respectively, generated by a virtual angiogram for the vascular model illustrated in FIGS. 7A-7E.

FIGS. 8A-8E are images of a vascular model for a ruptured aneurysm without contrast retention showing an inflow stream (FIG. 8A), flow pattern (FIG. 8B), vortex corelines (FIG. 8C), wall shear stress (FIG. 8D), and swirling (orange arrows 804) critical points.

FIGS. 8F-8G show selected frames at respective times (t₁-t₈) and time density curves, respectively, generated by a virtual angiogram for the vascular model illustrated in FIGS. 8A-8E.

### DETAILED DESCRIPTION

### General Considerations

For purposes of this description, certain aspects, advantages, and novel features of the embodiments of this disclosure are described herein. The disclosed methods and systems should not be construed as being limiting in any way. Instead, the present disclosure is directed toward all novel and nonobvious features and aspects of the various disclosed embodiments, alone and in various combinations and sub-combinations with one another. The methods and systems are not limited to any specific aspect or feature or combination thereof, nor do the disclosed embodiments require that any one or more specific advantages be present, or problems be solved. The technologies from any embodiment or example can be combined with the technologies described in any one or more of the other embodiments or examples. In view of the many possible embodiments to which the principles of the disclosed technology may be applied, it should be recognized that the illustrated embodiments are exemplary only and should not be taken as limiting the scope of the disclosed technology.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one skilled in the art.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person skilled in the art to have a more definitive construction, the numerical parameters set forth are approximations that may depend on the desired properties sought and/or limits of detection under standard test conditions/methods, as known to those skilled in the art. When directly and explicitly distinguishing embodiments from discussed prior art, the embodiment numbers are not approximates unless the word “about,” “substantially,” or “approximately” is recited. Whenever “substantially,” “approximately,” “about,” or similar language is explicitly used in combination with a specific value, variations up to and including 10% of that value are intended, unless explicitly stated otherwise.

Directions and other relative references may be used to facilitate discussion of the drawings and principles herein but are not intended to be limiting. For example, certain terms may be used such as “inner,” “outer,” “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part, and the object remains the same.

As used herein, “comprising” means “including,” and the singular forms “a” or “an” or “the” include plural references unless the context clearly dictates otherwise. The term “or” refers to a single element of stated alternative elements or a combination of two or more elements unless the context clearly indicates otherwise. As used herein, the use of “can” and “may” is intended to indicate that a particular recitation is optional.

Although there are alternatives for various components, parameters, operating conditions, etc. set forth herein, that does not mean that those alternatives are necessarily equivalent and/or perform equally well. Nor does it mean that the alternatives are listed in a preferred order, unless stated otherwise. Unless stated otherwise, any of the groups defined below can be substituted or unsubstituted.

Unless explained otherwise, all technical and scientific terms used herein have the same meaning as commonly understood to one skilled in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Features of the presently disclosed subject matter will be apparent from the following detailed description and the appended claims.

### Introduction

Disclosed herein are systems and methods for assessing one or more aneurysms in a patient via a virtual angiogram. As used herein, virtual angiogram refers to simulation transport of a contrast agent through a model of a portion of a vasculature of a patient based on a pulsatile flow velocity field. For the virtual angiogram, a patient-specific computational fluid dynamics (CFD) model of the vasculature portion can be constructed from 3D medical data of the patient. In some embodiments, in addition to providing visualization of the contrast as it flows through this model, the virtual angiogram can also be used to detect flow structures (e.g., flow stagnation, vortices, flow impingement areas, etc.) that may have detrimental effects on the aneurysm wall and/or induce growth, which could lead to eventual rupture. The detection of such flow structures may be helpful in assessing the likelihood of success of endovascular procedures used to treat aneurysms (e.g., to implant flow diverting stents) or to determine the possibility of thrombus formation within the aneurysm sac.

In some embodiments, one or more contrast retention metrics can be calculated based on the virtual angiogram. For example, each contrast retention metric can be based on a regional metric for the aneurysm compared to (e.g., ratio or difference) a corresponding regional metric for a parent artery. In some embodiments, the at least one of the contrast retention metrics can be compared to a predetermined threshold (or a predetermined range), and an aneurysm may be identified as unstable when the associated contrast retention metric is greater than the threshold (or outside the range). For example, aneurysm flow stagnation, which can be identified by contrast retention during angiography, is associated with aneurysm instability (growth or symptoms development) and can be used to identify aneurysms prone to further growth and potentially rupture. In some embodiments, such an identification may allow for early treatment of an aneurysm that would otherwise be at risk for growth and eventual rupture.

Alternatively or additionally, in some embodiments, the contrast retention metrics can be used to identify an aneurysm as ruptured, or at least potentially ruptured, for example, to discriminate between a plurality of aneurysms in a patient that requires treatment (e.g., emergency treatment when a patient presents with symptoms of a ruptured aneurysm). For example, the contrast retention metric for each of a plurality of aneurysms in the patient can be compared against each other, and the aneurysm having an extremum (e.g., maximum contrast retention metric) can be identified as ruptured. Alternatively or additionally, the contrast retention metric for each of a plurality of aneurysms can be compared against another predetermined threshold (or a predetermined range). Each aneurysm that has a contrast retention metric exceeding the threshold (or outside the range) may be identified as ruptured, or a score indicative of likelihood of rupture can be assigned based on the comparison with the threshold. Such an identification may allow for prompt treatment of the ruptured aneurysm when time may be of the essence.

In some embodiments, the aneurysm can be a cerebral aneurysm, such as a posterior communicating artery (PCOM) aneurysm. However, embodiments of the disclosed subject matter are not limited thereto. Rather, according to one or more contemplated embodiments, the systems and methods described herein can be used to assess (e.g., to identify as unstable and/or ruptured) other aneurysms in different parts of the body, such as thoracic or abdominal aortic aneurysms, aneurysms in renal arteries, or aneurysms in the arteries supplying blood to the legs.

### Aneurysm Assessment Methods

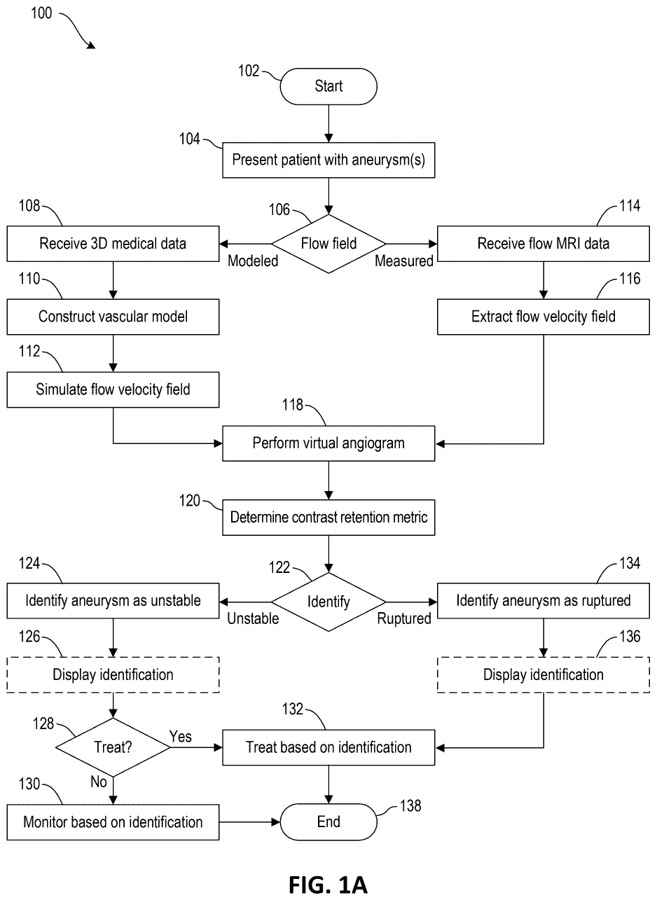

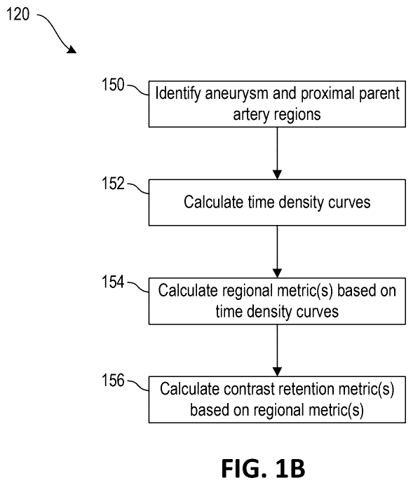

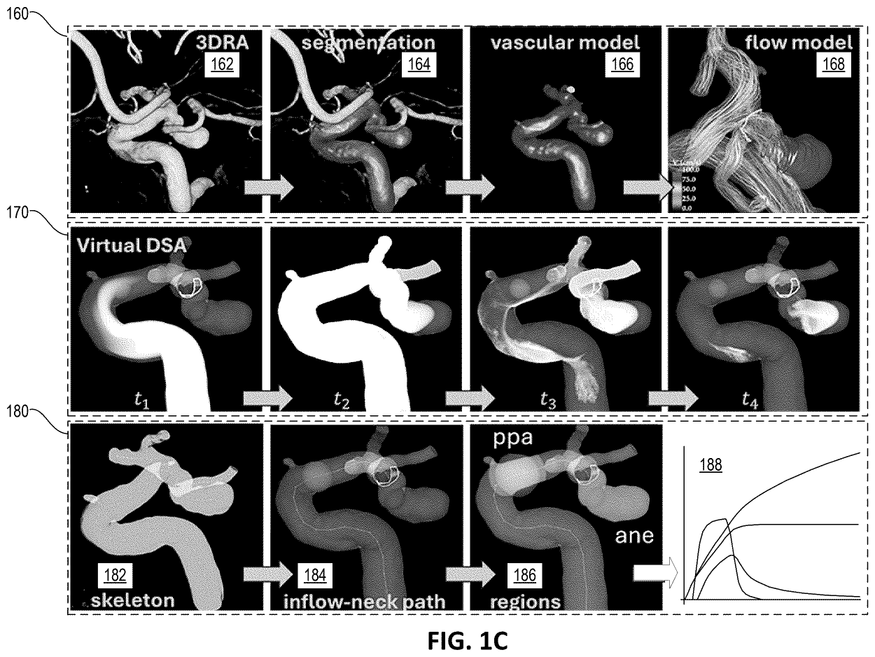

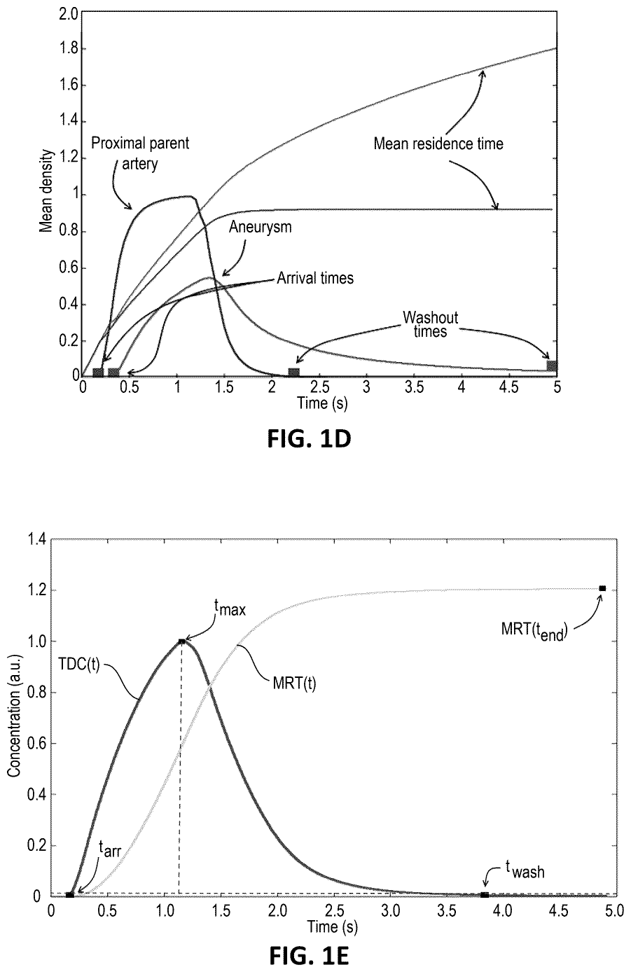

Referring to FIG. 1A, a method 100 for assessing an aneurysm is shown. The method 100 can initiate at terminator block 102 and proceed to process block 104, where a patient is presented that has one or more aneurysms. In some embodiments, the presentation of process block 104 is an emergent situation, for example, where the patient is exhibiting symptoms of a ruptured aneurysm. Alternatively, in some embodiments, the presentation of process block 104 is a non-emergent situation, for example, where the patient is being evaluated for potential treatment of an aneurysm prior to any rupture. In some embodiments, the presentation of process block 104 can involve a pre-screening of the patient to determine if a virtual angiogram should be performed. For example, medical imaging can be performed on the patient, and an aneurysm size, aneurysm location, and/or presence of blebs can be reviewed (e.g., similar to the review in clinical risk scoring, such as PHASES or ELAPSS). In some embodiments, if these factors do not imply significant risk, the method can proceed with a virtual angiogram for the patient; otherwise, if significant risk is implied, treatment may be recommended without performing a virtual angiogram. In some embodiments, the aneurysm being assessed is an intracranial aneurysm, such as a PCOM aneurysm. Alternatively or additionally, in some embodiments, the aneurysm is of a size that would normally not be considered for immediate interventional treatment, for example, having a maximum size of less than or equal to 7 mm.

The method 100 can proceed to decision block 106, where it is determined how the flow velocity field is obtained. In some embodiments, the flow velocity field is determined via computational fluid dynamics (CFD). In such embodiments, the method 100 can proceed from decision block 106 to perform process blocks 108-112. Row 160 in FIG. 1C illustrates an exemplary performance of process blocks 108-112. Alternatively, in some embodiments, the flow velocity field is directly measured in the vasculature of the patient. In such embodiments, the method 100 can proceed from decision block 106 to process block 114.

At process block 108, three-dimensional medical data of the patient's vasculature (or the portion thereof containing the aneurysm(s) of interest and connected parent vessels) can be received. In some embodiments, process block 108 can include subjecting the patient to one or more medical imaging modalities to obtain the medical data. In some embodiments, the three-dimensional medical data is obtained using a less-invasive imaging modality that does not employ intra-arterial catheterization, for example, a computed tomography angiogram (CTA) or magnetic resonance angiogram (MRA). Although CTA or MRA may utilize intravenous contrast administration, it is less invasive, faster, and safer than other angiogram modalities that require intra-arterial catheterization, such as three-dimensional rotational angiography (3DRA). Alternatively, in some embodiments, the medical data can be obtained using a more-invasive imaging modality (e.g., 3DRA) and/or a non-static imaging modality (e.g., 4D flow MRI). For example, image 162 in FIG. 1C reflects medical data obtained via 3DRA.

The method 100 can proceed to process block 110, where a model of the patient's vasculature can be constructed based at least in part on the obtained medical data. In some embodiments, aspects of construction of the vascular model (and subsequent CFD simulations of process block 112) may be similar to that described in Hadad et al., “CFD-based Virtual Angiograms for the Detection of Flow Stagnation in Intracranial Aneurysm,” Int J Numer Method Biomed Eng., August 2023, 39(8): e3740, which is incorporated by reference herein in its entirety. For example, images of the patient's vasculature can be segmented to construct a patient-specific 3D vascular model (e.g., as shown by image 164 in FIG. 1C), separating the aneurysm region from a defined proximal parent artery (PPA) region, and unstructured grids composed of volume elements (e.g., tetrahedral elements) can be generated to fill the volume of the vascular model (e.g., as shown by image 166 in FIG. 1C).

The method 100 can proceed to process block 112, where computational fluid dynamic (CFD) simulations of the constructed model can be performed for pulsatile flow conditions to obtain a patient-specific pulsatile flow velocity field (e.g., as shown by image 168 in FIG. 1C). For example, the CFD simulations can be conducted by numerically solving the Navier-Stokes equations under pulsatile flow conditions. The governing equations for the fluid flow are given by:

$$ρ ( \frac{\partial u}{\partial t} + u \cdot ∇ u ) = - ∇ p + μ {∇}^{2} u \qquad ( 1 )$$

$$∇ \cdot u = 0 \qquad ( 2 )$$

where u is the velocity vector, p is the pressure, ρ is the fluid density, and μ is the fluid viscosity. In some embodiments, subject-specific pulsatile inflow boundary conditions can be prescribed at the model inlet by scaling population-averaged flow waveforms, and blood can be approximated as an incompressible Newtonian fluid. In some embodiments, flow solutions can be obtained using a finite element solver. In some embodiments, the simulation data can include flow velocity fields stored at the mesh points at multiple instants of time during the cardiac cycle.

The method 100 can proceed to process block 118, where a virtual angiogram can be performed, for example, by simulating transport of contrast agent through the model of the patient's vasculature based on the patient-specific pulsatile flow velocity field (e.g., volume renderings at different times as shown in row 170 of FIG. 1C). In some embodiments, the simulating transport of the contrast agent can involve solving an advection transport equation over the computed velocity field, for example, in a manner similar to that described in Hadad et al., “CFD-based Virtual Angiograms for the Detection of Flow Stagnation in Intracranial Aneurysm,” Int J Numer Method Biomed Eng., August 2023, 39(8): e3740, incorporated by reference above. For example, to simulate the virtual angiogram and determine contrast retention, the dynamics of a contrast agent injected into the parent artery can be conducted by solving an advection or transport equation defined as:

$$\frac{\partial ϕ}{\partial t} + u \cdot ∇ ϕ = D {∇}^{2} ϕ + s \qquad ( 3 )$$

where φ represents the concentration of the contrast agent, D is the diffusivity, s is a production/destruction source term, and u is the fluid velocity obtained by solving the Navier-Stokes equations noted above. In some embodiments where only convection is considered (e.g., where the length is less than or equal to 10 cm and the velocity is less than or equal to 10 cm/s), the diffusivity and source terms can be set to zero (e.g., D=0, s=0).

In some embodiments, the convective velocity field can be linearly interpolated in time from the flow data stored during the cardiac cycle and can be assumed to be periodic from cycle to cycle. For example, a stepwise contrast injection with a duration of 1 second may be prescribed at the model inlet:

$${ϕ}_{i n} ( t ) = \{ \begin{matrix}1 & for t \leq 1 second \\ 0 & for t > 1 second\end{matrix} \qquad ( 4 )$$

where φᵢₙ is the inlet contrast concentration (e.g., in arbitrary units). In some embodiments where an in vivo injection profile is desired but unknown, a compartment model can be utilized to reconstruct the injection profile φᵢₙ(t). The compartment model can include a single compartment with a tracer concentration φ(t), a steady inflow and outflow Q equal to the mean flow into or out of the aneurysm, and a volume V corresponding to the aneurysm volume. For example, the governing equation for the compartment model can be given as:

$$V \frac{\partial ϕ}{\partial t} = Q {ϕ}_{i n} ( t ) - Q \overline{ϕ} \qquad ( 5 )$$

where φ̅ is the average concentration over the control volume. In some embodiments, the injection profile φᵢₙ(t) can be approximated with a gamma variate function (bolus equation) to describe the passage and dispersion of a bolus:

$${ϕ}_{i n} ( t ) = {Ate}^{- t / τ} \qquad ( 6 )$$

where the free parameters A (amplitude) and τ (injection duration) can be estimated to reproduce the time density curve.

Alternatively, when the method 100 proceeds from decision block 106 to process block 114, data can be received at process block 114 that is indicative of a direct measurement of the flow field in the patient. In some embodiments, process block 114 can include subjecting the patient to one or more medical imaging modalities to obtain the medical data. For example, the medical data of process block 114 can be measured via 4D Flow Magnetic Resonance Imaging (MRI) or Phase-Contrast MRI of the patient, which is a specialized scan that tracks the phase shift of protons in the blood and can provide a direct measurement of the speed and direction of the blood flowing through the patient's vasculature in three dimensions over time. At process block 116, the flow velocity field can be extracted from the data in process block 114. For example, in some embodiments, the 3D flow MRI scan can provide both 3D anatomical data (e.g., to see the shape of the aneurysm via magnitude information) and the flow velocity field (e.g., via phase shift information). Alternatively, in some embodiments, MRI anatomical data can be replaced by or supplemented by another imaging modality, for example, providing a higher resolution static scan such as CTA or 3DRA. In some embodiments, the 4D flow velocity data can be overlaid onto (e.g., co-registered with) the high-resolution 3D CTA model prior to or as part of the virtual angiogram. The method 100 can proceed from process block 116 to process block 118, where the virtual angiogram can be performed in a manner similar to that described above.

The method 100 can proceed from process block 118 to process block 120, where at least one contrast retention metric can be determined for the aneurysm based on the virtual angiogram. In some embodiments, the at least one contrast retention metric can be indicative of flow stagnation in the aneurysm. In some embodiments, the at least one retention metric can be based on regional metrics determined for a first vascular region (e.g., corresponding to the aneurysm) and a second vascular region (e.g., corresponding to a parent artery), for example, a difference or ratio between the regional metrics for the first and second regions. For example, in some embodiments, process block 120 may include one or more of process blocks 150-156, as shown in FIG. 1B.

At process block 150 of FIG. 1B, a first region in the model corresponding to the aneurysm and a second region in the model corresponding to a parent artery proximal to the aneurysm (PPA) can be identified. In some embodiments, to identify the aneurysm and PPA regions, a skeleton of the vascular model can be built, for example, as shown in image 182 of row 180 in FIG. 1C. The path from the aneurysm neck to the model inlet can then be identified by finding the skeleton points closest to the neck centroid and the inflow boundary, for example, as shown in image 184 of FIG. 1C. An algorithm can March 1 cm along this path from the aneurysm neck towards the inflow, and then mesh elements contained within a sphere having twice the vessel radius can be labeled, for example, as shown in image 186 of FIG. 1C.

At process block 152 of FIG. 1B, time density curves (TDCs) can then be calculated for contrast agent concentrations in each of the identified regions (e.g., as shown in graph 188 of FIG. 1C). For example, once the aneurysm region and the PPA region are labeled, the corresponding TDCs can be computed by averaging the contrast concentration over each respective region. An exemplary graph of the calculated TDCs is shown in FIG. 1D.

At process block 154 of FIG. 1B, at least one regional metric can be determined for each of the identified regions based on the calculated TDCs. For example, a Mean Residence Time (MRT) can be computed for each region to quantify stagnation based on the TDCs, where the MRT represents the transit time of the contrast through a specified region and is given by:

$$M R T = \frac{\int t {\overline{C}}^{i} ( t ) dt}{\int {\overline{C}}^{i} ( t ) dt} \qquad ( 7 )$$

where C̅ⁱ is the spatial average over region i for the contrast concentration. Alternatively or additionally, the regional metrics can include transit-time, time-to-peak, time-to-washout, and/or skewness. For example, for each region (i=ane, ppa), the time of the maximum concentration

$$( {t}_{m ax}^{i} )$$

can be found, and the arrival time

$$( {t}_{arr}^{i} )$$

and washout time

$$( {t}_{w s h}^{i} )$$

can be identified as the instants when the contrast crossed a threshold of 0.1% of the maximum concentration (e.g., as shown in FIG. 1E). From these values, the regional metrics can be calculated as transit-time:

$${TT}^{i} = {t}_{wsh}^{i} - {t}_{arr}^{i} ;$$

time-to-peak:

$${TP}^{i} = {t}_{m ax}^{i} - {t}_{arr}^{i} ;$$

time-to-washout:

$${TW}^{i} = {t}_{wsh}^{i} - {t}_{m ax}^{i} ;$$

and skewness: SKⁱ=TPⁱ/TWⁱ.

At process block 156, at least one contrast retention metric can be calculated based on the regional metrics from process block 154. In some embodiments, each contrast retention metric can be calculated as either (i) a difference between the regional metric for the first region and a corresponding regional metric for the second region, or (ii) a ratio of the regional metric for the first region and the corresponding regional metric for the second region. For example, the difference and ratio of each variable between the aneurysm and PPA can be respectively computed as: [symbol] (x)=x^{ane}−x^{ppa}, and [symbol] (x)=x^{ane}/x^{ppa} and used to quantify the contrast retention in the aneurysm with respect to the proximal parent artery. Such metrics can normalize the retention data against the PPA to isolate aneurysm-specific stagnation.

Return to FIG. 1A, the method 100 can then proceed from process block 120 to decision block 122, where the type of assessment is determined. In some embodiments, the purpose of the assessment may be to identify an aneurysm that has not yet ruptured but may be susceptible to future growth and subsequent rupture (e.g., unstable). In such embodiments, the method 100 can proceed from decision block 122 to perform blocks 124-132. Alternatively, in some embodiments, the purpose of the assessment is to discriminate a ruptured aneurysm from a plurality of aneurysms in the patient, for example, to target for emergency treatment. In such embodiments, the method 100 can proceed from decision block 106 to process block 134.

At process block 124, an aneurysm can be identified as unstable based at least in part on the at least one contrast retention metric. In some embodiments, the aneurysm can be identified as unstable responsive to the at least one contrast retention metric being greater than a predetermined threshold (or outside a predetermined range). In some embodiments, the predetermined threshold may comprise an optimal threshold (OT) statistically derived from patient cohort data to maximize diagnostic accuracy. For example, the OT may be determined by performing univariate or multivariate logistic regression analysis on a baseline dataset of known stable and unstable (e.g., growing or ruptured) aneurysms, and generating a Receiver Operating Characteristic (ROC) curve. The OT can correspond to the specific cutoff value on the ROC curve that provides the optimal mathematical balance between sensitivity (true positive rate) and specificity (true negative rate), such as by maximizing the Youden Index. In some embodiments, the OT for identifying an unstable or growing aneurysm may be defined based on an area under the curve (AUC) analysis. For example, in some embodiments, an aneurysm can be identified as unstable when [symbol] (TT) is greater than 0.72 and/or when [symbol] (TW) is greater than 0.68 and/or when R(TT) is greater than 1.79. Alternatively or additionally, the aneurysm can be identified as unstable when [symbol] (MRT) is greater than another threshold. In some embodiments, a threshold for use in comparison with the contrast retention metric(s) can be determined from, or at least based on, multivariate logistic regression. Alternatively or additionally, in some embodiments, the contrast retention metric(s) can be used by machine learning techniques or classifiers (e.g., random forests) to identify an aneurysm as stable, unstable, or ruptured, with or without determining particular thresholds.

The method 100 can proceed to optional process block 126, where the aneurysm identification can be conveyed to a system user and/or medical personnel, for example, via a display. In some embodiments, process block 126 can include displaying the identified assessment (e.g., stable or unstable) for the aneurysm and/or the underlying contrast retention metric or other predictive risk score underlying the identification. In some embodiments, the display of process block 126 can take the form of a graphical user interface, for example, overlaying the calculated contrast retention metric onto a visualization of the 3D vascular model, for example, to guide a physical treatment intervention.

The method 100 can proceed to decision block 128, where it is determined if an interventional treatment should be performed (e.g., physically administered) based on the identification. For example, responsive to identifying the aneurysm as unstable, the method 100 can proceed to process block 132, where the patient is subjected to an interventional treatment prior to rupture of the aneurysm. As used herein, an “interventional treatment” refers to any active physical or localized therapeutic procedure designed to stabilize, occlude, or obliterate the aneurysm, including but not limited to endovascular procedures (e.g., coiling, flow diversion), surgical procedures (e.g., microsurgical clipping), and non-invasive radiosurgical procedures (e.g., stereotactic radiosurgery). Alternatively, if the aneurysm is identified as stable, the method 100 can proceed from decision block 128 to process block 130, where the patient may otherwise be subject to surveillance (e.g., to periodically monitor the aneurysm for changes) and/or other treatments of a non-interventional nature (e.g., patient lifestyle management).

Alternatively, when the method 100 proceeds from decision block 122 to process block 134, an aneurysm can be identified as ruptured based at least in part on the at least one contrast retention metric. In some embodiments, the ruptured aneurysm may be one of a plurality of aneurysms in the patient. For example, when a patient suffers a subarachnoid hemorrhage (bleeding in the brain) and is rushed to the hospital, scans often reveal that the patient has multiple intracranial aneurysms. Because the bleeding floods the brain space, standard imaging often cannot definitively show which of the aneurysms actually burst. In some embodiments, the identification of process block 134 can avoid, or at least reduce the likelihood of, treating the unruptured aneurysm while leaving the ruptured one alone, which could otherwise put the patient at imminent risk of a fatal re-bleed. In some embodiments, at least one of the plurality of aneurysms in the patient can be identified as ruptured responsive to the corresponding contrast retention metric being greater than a predetermined threshold. Alternatively, in some embodiments, the contrast retention metrics for each of the plurality of aneurysms can be compared against each other, and the aneurysm having an extremum for the contrast retention metric (e.g., the highest value) being identified as ruptured.

The method 100 can proceed to optional process block 136, where the aneurysm identification can be conveyed to a system user and/or medical personnel, for example, via a display. In some embodiments, process block 136 can include displaying an indication of which aneurysm has ruptured and/or the underlying contrast retention metric or other predictive risk score underlying the identification. In some embodiments, the display of process block 136 can take the form of a graphical user interface, for example, overlaying the calculated contrast retention metric onto a visualization of the 3D vascular model, for example, to guide a physical treatment intervention.

After process block 134 and/or process block 136, the method 100 can proceed to process block 132, where the patient is subjected to an interventional treatment, for example, to stabilize, occlude, or obliterate the aneurysm identified as ruptured. After process block 130 and/or process block 132, the method 100 can end at terminal block 138.

Although blocks 102-138, 150-156 of FIGS. 1A-1B have been described as being performed once, in some embodiments, multiple repetitions of a particular block may be employed before proceeding to the next decision block or process block. In addition, although blocks 102-138, 150-156 of FIGS. 1A-1B have been separately illustrated and described, in some embodiments, process blocks may be combined and performed together (simultaneously or sequentially). Moreover, although FIGS. 1A-1B illustrate a particular order for blocks 102-138, 150-156, embodiments of the disclosed subject matter are not limited thereto. Indeed, in certain embodiments, the blocks may occur in a different order than illustrated or simultaneously with other blocks. In some embodiments, method 100 and/or process block 120 can include steps or other aspects not specifically illustrated in FIGS. 1A-1B. Alternatively or additionally, in some embodiments, method 100 and/or process block 120 may comprise only some of blocks 102-138, 150-156 of FIGS. 1A-1B.

### Aneurysm Assessment Systems

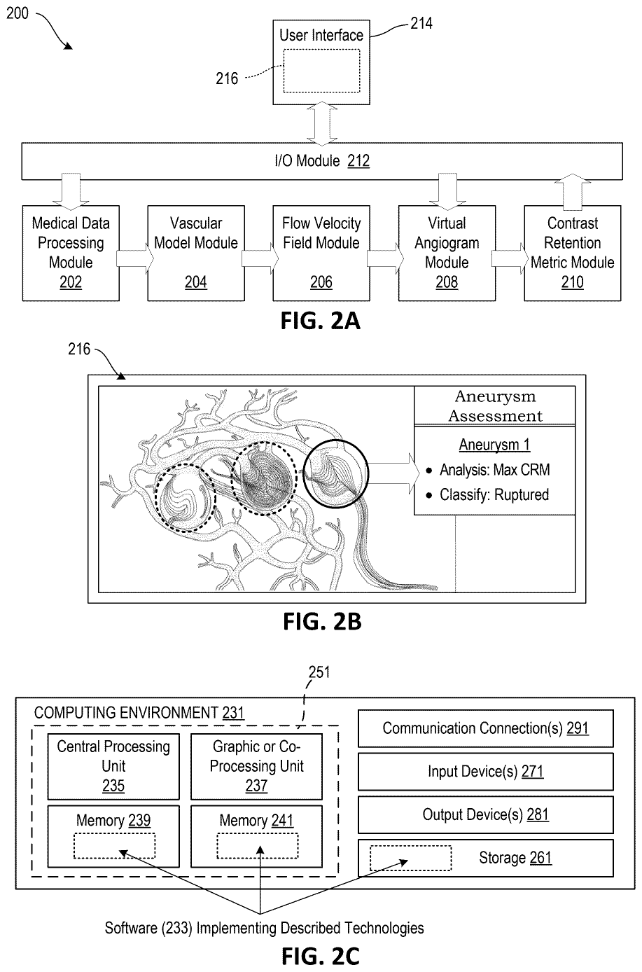

FIG. 2A shows an example configuration for an aneurysm assessment system 200. In the illustrated example, system 200 includes a medical data processing module 202, a vascular model module 204, a flow velocity field module 206, a virtual angiogram module 208, a contrast retention metric module 210, an input/output (I/O) module 212, and a user interface 214. However, fewer or more modules may be provided in other systems according to one or more contemplated embodiments. Moreover, although certain functions and operations are discussed below as being associated with particular modules, functions/operations can instead be performed by other modules and/or be shared between modules according to one or more contemplated embodiments. Accordingly, embodiments of the disclosed subject matter are not limited to the specific configuration illustrated in FIG. 2A.

In some embodiments, the medical data processing module 202 can be configured to receive (e.g., via I/O module 212) and/or process 3D medical data of a patient. For example, the 3D medical data can be obtained via CTA or MRA of the patient. Alternatively or additionally, the 3D medical data can be obtained via 3DRA or 4D flow MRI. In some embodiments, the medical data processing module 202 can configure the 3D medical data for use by vascular model module 204. In some embodiments, the vascular model module 204 can construct a model of a portion of the vasculature of the patient based at least in part on the 3D medical data. For example, the vascular model module 204 can be configured to construct the model in a manner similar to that described above for process block 110 in FIG. 1A and/or images 164-166 in FIG. 1C.

In some embodiments, the vascular model module 204 can provide the constructed model to the flow velocity field module 206. In some embodiments, the flow velocity field module 206 can perform one or more CFD simulations of the constructed model for pulsatile flow conditions to obtain a patient-specific pulsatile flow velocity field. For example, the flow velocity field module 206 can be configured to perform the CFD simulation(s) in a manner similar to that described above for process block 112 in FIG. 1A and/or image 168 in FIG. 1C. In some embodiments, the flow velocity field module 206 can provide the patient-specific pulsatile flow velocity field to the virtual angiogram module 208. Alternatively or additionally, in some embodiments, the virtual angiogram module 208 can directly receive (e.g., via I/O module 212) data indicative of an actual pulsatile flow velocity field for the patient, for example, when measured using 4D flow MRI.

In some embodiments, the virtual angiogram module 208 can perform a virtual angiogram by simulating transport of contrast agent through the constructed model based on the patient-specific pulsatile flow velocity field. For example, the virtual angiogram module 208 can be configured to perform the virtual angiogram in a manner similar to that described above for process block 118 in FIG. 1A and/or row 170 in FIG. 1C. In some embodiments, the virtual angiogram module 208 can provide data from the virtual angiogram (e.g., time density curves (TDCs), or data used to calculate such TDCs) to the contrast retention metric module 210.

In some embodiments, the contrast retention metric module 210 can determine at least one contrast retention metric for an aneurysm based on the provided data from the virtual angiogram. In some embodiments, the contrast retention metric module 210 can identify an aneurysm as stable, unstable, or ruptured based at least in part on the at least one contrast retention metric. In some embodiments, the contrast retention metric module 210 can identify regions in the model corresponding to the aneurysm and a parent artery proximal to the aneurysm, and can calculate TDCs for contrast agent concentration in each of the identified regions. In some embodiments, the contrast retention metric module 210 can determine at least one regional metric for each of the identified regions based on the calculated TDCs, and can calculate a contrast retention metric as (i) a difference between the regional metrics for the identified regions, or (ii) a ratio of the regional metrics for the identified regions. For example, the contrast retention metric module 210 can be configured to determine the contrast retention metric in a manner similar to that described above for process block 120 in FIG. 1A, process blocks 150-152 in FIG. 1B, and/or row 180 in FIG. 1C. Alternatively or additionally, the contrast retention metric module 210 can perform the identification of the aneurysm as stable, unstable, or ruptured in a manner similar to that described above for process block 124 and/or process block 134 in FIG. 1A.

In some embodiments, the contrast retention metric module 210 can be configured to provide an output (e.g., via I/O module 212), for example, to drive a display 216 of user interface 214. In some embodiments, the display 216 may include an overlay alerting a system operator or other medical personnel of the aneurysm identified (e.g., ruptured or unstable), for example, as shown in FIG. 2B. In some embodiments, this automated classification of the aneurysm can directly guide the physical administration of an interventional treatment (e.g., endovascular procedure, a surgical clipping, or a stereotactic radiosurgical ablation), thereby preemptively treating an unstable aneurysm before a catastrophic rupture event occurs or efficiently treating a ruptured aneurysm.

In some embodiments, software components, applications, routines or sub-routines, or sets of instructions for causing one or more processors to perform certain functions may be referred to as “modules” or “engines.” It should be noted that such modules or engines, or any software or computer program referred to herein, may be written in any computer language and may be a portion of a monolithic code base, or may be developed in more discrete code portions, such as is typical in object-oriented computer languages. In addition, the modules or engines, or any software or computer program referred to herein, may in some embodiments be distributed across a plurality of computer platforms, servers, terminals, and the like. For example, a given module or engine may be implemented such that the described functions are performed by separate processors and/or computing hardware platforms. Further, although certain functionality may be described as being performed by a particular module or engine, such description should not be taken in a limiting fashion. In other embodiments, functionality described herein as being performed by a particular module or engine may instead (or additionally) be performed by a different module, engine, program, sub-routine or computing device without departing from the spirit and scope of the invention(s) described herein.

It should be understood that any of the software modules, engines, or computer programs illustrated herein may be part of a single program or integrated into various programs for controlling one or more processors of a computing device or system. Further, any of the software modules, engines, or computer programs illustrated herein may be stored in a compressed, uncompiled, and/or encrypted format and include instructions which, when performed by one or more processors, cause the one or more processors to operate in accordance with at least some of the methods described herein. Of course, additional and/or different software modules, engines, or computer programs may be included, and it should be understood that the examples illustrated and described with respect to the figures herein are not necessary in any embodiments. Use of the terms “module” or “software engine” is not intended to imply that the functionality described with reference thereto is embodied as a stand-alone or independently functioning program or application. While in some embodiments functionality described with respect to a particular module or engine may be independently functioning, in other embodiments such functionality is described with reference to a particular module or engine for ease or convenience of description only and such functionality may in fact be a part of, or integrated into, another module, engine, program, application, or set of instructions for directing a processor of a computing device.

In some embodiments, the instructions of any or all of the software modules, engines or programs described above may be read into a main memory from another computer-readable medium, such from a read-only memory (ROM) to random access memory (RAM). Execution of sequences of instructions in the software module(s) or program(s) can cause one or more processors to perform at least some of the processes or functionalities described herein. Alternatively or additionally, in some embodiments, hard-wired circuitry may be used in place of, or in combination with, software instructions for implementation of the processes or functionalities described herein. Thus, the embodiments described herein are not limited to any specific combination of hardware and software.

### Computer Implementation Examples

FIG. 2C depicts a generalized example of a suitable computing environment 231 in which the described innovations may be implemented, such as but not limited to aspects of method 100 and/or aneurysm assessment system 200 (or one or more components thereof). The computing environment 231 is not intended to suggest any limitation as to scope of use or functionality, as the innovations may be implemented in diverse general-purpose or special-purpose computing systems. For example, the computing environment 231 can be any of a variety of computing devices (e.g., desktop computer, laptop computer, server computer, tablet computer, etc.).

With reference to FIG. 2C, the computing environment 231 includes one or more processing units 235, 237 and memory 239, 241. In FIG. 2C, this basic configuration 251 is included within a dashed line. The processing units 235, 237 execute computer-executable instructions. A processing unit can be a central processing unit (CPU), processor in an application-specific integrated circuit (ASIC), or any other type of processor (e.g., hardware processors, graphics processing units (GPUs), virtual processors, etc.). In a multi-processing system, multiple processing units execute computer-executable instructions to increase processing power. For example, FIG. 2C shows a central processing unit 235 as well as a graphics processing unit or co-processing unit 237. The tangible memory 239, 241 may be volatile memory (e.g., registers, cache, RAM), non-volatile memory (e.g., ROM, EEPROM, flash memory, etc.), or some combination of the two, accessible by the processing unit(s). The memory 239, 241 stores software 233 implementing one or more innovations described herein, in the form of computer-executable instructions suitable for execution by the processing unit(s).

A computing system may have additional features. For example, the computing environment 231 includes storage 261, one or more input devices 271, one or more output devices 281, and one or more communication connections 291. An interconnection mechanism (not shown) such as a bus, controller, or network interconnects the components of the computing environment 231. Typically, operating system software (not shown) provides an operating environment for other software executing in the computing environment 231, and coordinates activities of the components of the computing environment 231.

The tangible storage 261 may be removable or non-removable, and includes magnetic disks, magnetic tapes or cassettes, CD-ROMs, DVDs, or any other medium which can be used to store information in a non-transitory way, and which can be accessed within the computing environment 231. The storage 261 can store instructions for the software 233 implementing one or more innovations described herein.

The input device(s) 271 may be a touch input device such as a keyboard, mouse, pen, or trackball, a voice input device, a scanning device, or another device that provides input to the computing environment 231. The output device(s) 281 may be a display, printer, speaker, CD-writer, or another device that provides output from computing environment 231.

The communication connection(s) 291 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, radio-frequency (RF), or another carrier.

Any of the disclosed methods can be implemented as computer-executable instructions stored on one or more computer-readable storage media (e.g., one or more optical media discs, volatile memory components (such as DRAM or SRAM), or non-volatile memory components (such as flash memory or hard drives)) and executed on a computer (e.g., any commercially available computer, including smart phones or other mobile devices that include computing hardware). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or any other such network) using one or more network computers.

For clarity, only certain selected aspects of the software-based implementations are described. Other details that are well known in the art are omitted. For example, it should be understood that the disclosed technology is not limited to any specific computer language or program. For instance, aspects of the disclosed technology can be implemented by software written in C++, Java™, Python®, and/or any other suitable computer language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means. In any of the above-described examples and embodiments, provision of a request (e.g., data request), indication (e.g., data signal), instruction (e.g., control signal), or any other communication between systems, components, devices, etc. can be by generation and transmission of an appropriate electrical signal by wired or wireless connections.

### Aneurysm Assessment Examples

### Example 1

This study compared stagnation properties of stable, unstable, and ruptured aneurysms at a single location, the posterior communicating artery (PCOM), to control for location specific characteristics. This is an important cohort because aneurysms at this location have a relatively large rupture risk, and patients can develop severe symptoms related to cerebral nerve III (CN III) palsy, which can be considered a sign of aneurysm instability. A total of 183 cerebral aneurysms located at the PCOM were studied. Of these, 23 were previously classified as stable after at least one year of follow up without treatment and with no appreciable change in size or shape. Another 18 were classified as unstable, either because they were observed to grow by at least 0.5 mm during follow up (n=13) or had developed CN III palsy (n=5). The rest (n=142) were PCOM aneurysms that had been presented as ruptured.

Patient-specific vascular models were constructed from 3D rotational angiography (3DRA) images. Corresponding computational fluid dynamics (CFD) simulations were conducted by numerically solving the Navier-Stokes equations under pulsatile inflow conditions derived from population averaged waveforms scaled with the patient-specific inflow vessel area using an empirical relationship. Flow solutions were found with a finite element solver on unstructured grids with a minimum resolution of 200 μm. The simulation data included flow velocity fields stored at the mesh points at 100 instants of time during the cardiac cycle.

Virtual angiograms were performed by simulating transport of a contrast agent injected at the inlet, in particular, by numerically solving the advection equation using an explicit finite volume approach as described in Hadad et al., “CFD-based Virtual Angiograms for the Detection of Flow Stagnation in Intracranial Aneurysm,” Int J Numer Method Biomed Eng., August 2023, 39(8): e3740, incorporated by reference above. The convective velocity field was linearly interpolated in time from the flow data stored at 100 instants during the cardiac cycle and it was assumed to be periodic from cycle to cycle. A stepwise contrast injection with a duration of 1 second was prescribed at the model inlet:

$${C}_{0} ( t ) = \{ \begin{matrix}1 & t \leq 1 sec \\ 0 & t > 1 sec\end{matrix}$$

where C₀ is the inlet contrast concentration (in arbitrary units). The transport equation was advanced in time until the maximum contrast concentration over the entire vascular model fell below a specified threshold of the maximum over time. The threshold was set at 0.1%, which typically required less than 5 seconds (e.g., 5 cardiac cycles) to wash out the entire vascular model. The contrast concentration field was saved at 0.05 sec intervals (equivalent to a 20 frames per second angiogram). Visualizations of the contrast field were created by rendering the mesh elements with transparency and gray values modulated by the contrast concentration.

To quantify the contrast retention of the aneurysm with respect to the parent artery, time density curves were constructed by computing the average contrast concentration in the corresponding regions. The aneurysm region was identified by computing the path distance between tetrahedral mesh elements and the aneurysm neck surface (e.g., oriented surface triangulation through the aneurysm orifice). To identify the proximal parent artery region, a skeleton of the vascular model was built, and a path from the aneurysm neck to the model inlet was identified by finding the skeleton points closest to the neck centroid and the inflow boundary. The process marched 1 cm along this path from the aneurysm neck towards the inflow, and mesh elements contained within a sphere with twice the vessel radius were labeled.

Once the aneurysm (ane) and proximal parent artery (ppa) regions were identified and labeled, the corresponding time density curves were computed. For each region (i=ane, ppa), the arrival time

$$( {t}_{arr}^{i} )$$

and washout time

$$( {t}_{wsh}^{i} )$$

were identified as the instants when the contrast crossed a threshold of 0.1% of the curve maximum, and from these the total transit time were defined:

$${TT}^{i} = {t}_{wsh}^{i} - {t}_{arr}^{i} .$$

The transit time ratio (R(TT)) between the aneurysm and proximal parent artery transit times (e.g., R(TT)=TT^{ane}/TT^{ppa}) was used as a metric for contrast retention within the aneurysm. A second contrast retention metric was calculated as the ratio between the mean residence times (MRT) of the aneurysm over the proximal parent artery region (e.g., R(MRT)=MRT^{ane}/MRT^{ppa}, where the mean residence time in region i is defined as MRTⁱ=∫tC̅ⁱ(t)dt/∫C̅ⁱ(t)dt, and C̅ⁱ the spatial average over region i.

The average contrast retention time ratio of the aneurysm over the proximal parent artery regions (quantified through R(TT) and R(MRT)) were statistically compared between stable and unstable aneurysms, between stable and ruptured aneurysms, and between stable and a combination of unstable and ruptured aneurysms, using the non-parametric Wilcoxon (Mann-Whitney) test. Furthermore, univariate logistic regression (LR) models were fitted and the corresponding odds-ratio (OR), area under the receiving operating characteristics (ROC) curves (AUC), and optimal thresholds (OT) were calculated. Differences with a p-value<0.05 were considered statistically significant. Since the set of ruptured aneurysms was substantially larger (n=142) than the stable aneurysm set (n=23), a down-sampling approach was used by randomly selecting (23) ruptured aneurysms and performing 1000 bootstrap repetitions of the Wilcoxon tests and the univariate models to calculate the mean p-values, odds-ratios, AUCs, and optimal thresholds.

TABLE 1
Comparison of contrast total transit time ratio (R(TT)) and mean residence
time ratio (R(MRT)) of an aneurysm over parent artery regions between stable
and unstable and/or ruptured PCOM aneurysms, where < > indicated
average over 1000 down-sampling bootstrap repetitions, OR represents odds-
ratio, AUC represents the area under the curve, and OT represents optimal threshold.

| | | | Wilcoxon test | | Univariate Logistic Regression | | | |
|---|---|---|---|---|---|---|---|---|
| Variable | Group | N | Mean ± SD | p | OR | p | AUC | OT |
| R(TT) | Stable | 23 | 1.3034 ± 0.3036 | — | — | — | — | — |
| | Unstable | 18 | 1.8128 ± 0.7262 | 0.0235 | 6.64 | 0.0147 | 0.71 | 1.79 |
| | Ruptured | 142 | 1.6182 ± 0.6137 | 0.0353 | 3.70 | 0.0249 | 0.64 | 2.96 |
| | Combined | 160 | 1.6401 ± 0.6278 | 0.0247 | 3.84 | 0.0195 | 0.65 | 2.99 |
| R(MRT) | Stable | 23 | 1.2776 ± 0.3073 | — | — | — | — | — |
| | Unstable | 18 | 1.5965 ± 0.4404 | 0.0043 | 13.34 | 0.0264 | 0.76 | 1.74 |
| | Ruptured | 142 | 1.4038 ± 0.3918 | 0.0382 | 4.41 | 0.1414 | 0.64 | 3.54 |
| | Combined | 160 | 1.4255 ± 0.4008 | 0.0212 | 5.45 | 0.0914 | 0.65 | 3.61 |

Down-sampling Bootstrap Repetitions

| | | | <Mean> ± <SD> | <p> | <OR> | <p> | <AUC> | <OT> |
|---|---|---|---|---|---|---|---|---|
| R(TT) | Ruptured | 23 | 1.1618 ± 0.6085 | 0.1916 | 4.76 | 0.0937 | 0.64 | 1.91 |
| | Combined | 41 | 1.7039 ± 0.6656 | 0.0433 | 4.95 | 0.0229 | 0.67 | 2.28 |
| R(MRT) | Ruptured | 23 | 1.4019 ± 0.3617 | 0.1849 | 3.86 | 0.3151 | 0.64 | 1.91 |
| | Combined | 41 | 1.4873 ± 0.4141 | 0.0199 | 8.19 | 0.0609 | 0.69 | 2.22 |

Comparisons of the transit time ratio (R(TT)) between stable, unstable, and ruptured aneurysms are presented in Table 1. It can be seen that in general unstable aneurysms had larger transit time ratios (e.g., more flow stagnation) than stable aneurysms (p=0.0235, OR=6.64). Furthermore, R(TT) was a good discriminator between stable and unstable aneurysms (AUC=0.71, OT=1.79). Similar comparisons for mean residence time ratios (R(MRT)) are also presented in Error! Reference source not found. R(MRT) also had a larger value in unstable than in stable aneurysms (p=0.0043, OR=13.34) and was a slightly better discriminator between these aneurysm types (AUC=0.76, OT=1.74).

Compared to stable aneurysms, ruptured aneurysms tended to have larger R(TT) and R(MRT), but these associations, on average, did not reach statistical significance when performing bootstrap repetitions with down-sampling (e.g., <p>=0.1916 and <p>=0.1849, respectively). Univariate LR showed that, on average, both R(TT) and R(MRT) were fair discriminators between stable and ruptured aneurysms, with an average <AUC>=0.64 and mean optimal threshold <OT>=1.91. On the other hand, when combining ruptured and unstable aneurysms, they had on average more contrast retention than stable aneurysms (R(TT): <p>=0.0433, R(MRT): <p>=0.0199) and both R(TT) and R(MRT) were able to discriminate between these groups with fair accuracy (e.g., R(TT): <AUC>=0.67, <OT>=2.28; R(MRT): <AUC>=0.69, <OT>=2.22).

The results of this study suggest that PCOM aneurysm growth and/or development of symptoms related to cranial nerve palsy are associated or favored by hemodynamic environments characterized by slow and stagnant flows. These kinds of aneurysm flows environments can be recognized by the retention of contrast in angiographic images, either real digital subtraction angiography (DSA) sequences or virtual angiograms based on CFD models. As such, contrast retention can be considered a risk factor for aneurysm instability (growth and/or symptoms development), which in itself is a risk factor for aneurysm rupture. Although, in general, stable aneurysms had less flow stagnation, the absence of contrast retention should not be considered an indication of low rupture risk, since many ruptured aneurysms did not exhibit significant contrast retention. In the absence of contrast retention, other flow conditions and rupture risk factors (e.g., strong impingements, vortex flows, complex and unstable flow patterns, etc.) should be further analyzed to assess the likelihood of aneurysm rupture without stagnation.

### Example 2

PCOM aneurysms were modeled with image-based CFD to form two subsets: a) cross-sectional dataset with a total of 271 PCOM aneurysms including 129 unruptured (U) aneurysms and 142 ruptured (R) aneurysms, and b) a longitudinal dataset with a total of 41 PCOM aneurysms, including 23 stable(S) aneurysms that showed no changes in serial imaging, and 13 aneurysms that exhibited enlargement in follow-up images along with 5 aneurysms that developed CNIII palsy which were combined into a growing (G) aneurysm group with 18 aneurysms. These cross-sectional and longitudinal datasets were used to investigate associations between aneurysm rupture and instability with intra-aneurysmal flow stagnation quantified from virtual angiograms created from the CFD simulations.

Patient-specific vascular models were constructed by segmentation of 3D rotational angiography (3DRA) images, and corresponding CFD simulations were conducted by numerically solving the Navier-Stokes equations under pulsatile flow conditions. Inflow boundary conditions were derived from population averaged waveforms scaled with the patient-specific inflow vessel area using an empirical relationship. Flow solutions were obtained with a finite element solver on unstructured grids with a minimum resolution of 200 μm. The simulation data included flow velocity fields stored at the mesh points at 100 instants of time during the cardiac cycle.

Virtual angiograms were performed by simulating transport of a contrast agent injected at the inlet, in particular, by numerically solving the advection equation using an explicit finite volume approach as described in Hadad et al., “CFD-based Virtual Angiograms for the Detection of Flow Stagnation in Intracranial Aneurysm,” Int J Numer Method Biomed Eng., August 2023, 39(8): e3740, incorporated by reference above. The convective velocity field was linearly interpolated in time from the flow data stored at 100 instants during the cardiac cycle, and it was assumed to be periodic from cycle to cycle. A stepwise contrast injection with a duration of 1 second was prescribed at the model inlet:

$${C}_{0} ( t ) = \{ \begin{matrix}1 & t \leq 1 sec \\ 0 & t > 1 sec\end{matrix}$$

where C₀ is the inlet contrast concentration (in arbitrary units). The transport equation was advanced in time until the maximum contrast concentration over the entire vascular model fell below a specified threshold. The threshold was prescribed at 0.1%, of the maximum concentration which typically required less than 5 second (e.g., 5 cardiac cycles) to wash out the entire vascular model. The contrast concentration field was saved at 0.05 second intervals (equivalent to a 20 fps angiogram). Visualizations of the contrast field were created by rendering the mesh elements with transparency and gray values modulated by the contrast concentration.

To quantify the contrast retention of the aneurysm with respect to the parent artery, time density curves were constructed by computing the average contrast concentration in corresponding regions. The aneurysm region was identified by computing the path distance between tetrahedral mesh elements and the aneurysm neck surface (e.g., oriented surface triangulation through the aneurysm orifice). To identify the proximal parent artery region, a skeleton of the vascular model was built, and a path from the aneurysm neck to the model inlet was identified by finding the skeleton points closest to the neck centroid and the inflow boundary. The process then marched 1 cm along this path from the aneurysm neck towards the inflow, and mesh elements contained within a sphere with twice the vessel radius were labeled.

Once the aneurysm (ane) and proximal parent artery (ppa) regions were identified and labeled, the corresponding time density curves were computed by averaging the contrast concentration over the region. For each region (i=ane, ppa), the time of the maximum concentration

$$( {t}_{ma x}^{i} )$$

was found, and the arrival time

$$( {t}_{arr}^{i} )$$

and washout time

$$( {t}_{wsh}^{i} )$$

were identified as the instants when the contrast crossed a threshold of 0.1% of the maximum concentration. From these values the following regional metrics were calculated:

$$\text{transit-time:} {TT}^{i} = {t}_{wsh}^{i} - {t}_{arr}^{i} ; \\ \text{time-to-peak:} {TP}^{i} = {t}_{m ax}^{i} - {t}_{arr}^{i} ; \\ \text{time-to-washout:} {TW}^{i} = {t}_{wsh}^{i} - {t}_{m ax}^{i} ; \\ \text{skewness:} {SK}^{i} = {TP}^{i} / {TW}^{i} ; \\ and \\ \text{mean-residence-time:} {MRT}^{i} = \int t {\overline{C}}^{i} ( t ) dt / \int {\overline{C}}^{i} ( t ) dt , \\ where {\overline{C}}^{ι} \text{is the spatial average over region} i .$$

Based on the regional metrics, contrast retention metrics were calculated, for example, as the difference and ratio of each regional metric (x) between the aneurysm and parent artery were respectively computed as: [symbol] (x)=x^{ane}−x^{ppa}, and [symbol] (x)=x^{ane}/x^{ppa}. These calculated metrics were then used to quantify the contrast retention in the aneurysm with respect to the proximal parent artery, and to provide quantitative assessments of aneurysm flow stagnation. The average contrast retention metrics were statistically compared between unruptured and ruptured aneurysms from the cross-sectional dataset, as well as between stable and growing aneurysms from the longitudinal dataset, using the non-parametric Wilcoxon (Mann-Whitney) test. The unruptured versus ruptured comparisons were repeated for small aneurysms (e.g., maximum aneurysm size less than or equal to 7 mm). Differences with a p-value<0.05 were considered statistically significant. Univariate logistic regression (LR) models were fitted for each metric and the corresponding odds-ratio (OR) and area under the receiving operating characteristics (ROC) curves (AUC) were calculated.

Associations between flow stagnation and aneurysm rupture and instability were further analyzed by first classifying aneurysms flows into two categories corresponding to stagnation and no-stagnation. Aneurysms with [symbol] (TT)>0.72 or [symbol] (TW)>0.68 were classified as having stagnation. Once categorized, flow stagnation was compared with aneurysm rupture and instability using 2×2 contingency table analysis and the Fisher exact test. Finally, multivariate logistic regression models were fitted to the datasets. All contrast retention metrics were included in the initial models and stepwise variable selection based on the Akaike's Information Criterion (AIC) was conducted, and the AUC and the cross-validation error based on the leave-one-out method were calculated for the final models.

Comparisons of the difference ( [symbol] ) and ratio ( [symbol] ) of contrast retention metrics (TT, TP, TW, SK, MRT) between the aneurysm and parent artery regions for ruptured and unruptured aneurysms (all and smaller than 7 mm) as well as stable and growing aneurysms are presented in Table 2. Values in each group are listed as mean±standard deviation. The p-values correspond to the Mann-Whitney test, and the odds ratios with their 95% confidence intervals and the AUCs were obtained from univariate logistic regressions. Statistically significant differences (95% confidence) are indicated with a ‘*’.

Several metrics indicative of longer retention in the aneurysm compared to the parent artery (e.g., [symbol] (TT), [symbol] (TT), [symbol] (TW), [symbol] (TW), [symbol] (MRT)) were significantly larger in ruptured aneurysms as compared to unruptured aneurysms from the cross-sectional dataset; however, their discriminatory power as quantified by the AUC was only moderate (e.g., with largest values between 0.61 and 0.64). Similar results, with slightly larger p-values, were obtained when restricting the analysis to aneurysms smaller than 7 mm. On the other hand, stronger associations with larger odds ratios and good discriminatory power (e.g., max AUC between 0.70-0.77) were obtained when comparing stable and growing aneurysms from the longitudinal dataset.

After classifying aneurysms into flow stagnation categories (stagnation=Yes, stagnation=No) by thresholding the aneurysm-artery difference in transit-time (TT) and washout-time (TW), the number of ruptured/unruptured and stable/growing aneurysms in each category were counted and compared. As suggested by the results in Table 3, flow stagnation is more prevalent in ruptured and growing aneurysms compared to unruptured or stable aneurysms, respectively. However, due to the smaller sample size of the longitudinal dataset, the association between flow stagnation and aneurysm instability only reached marginal significance (94% confidence).

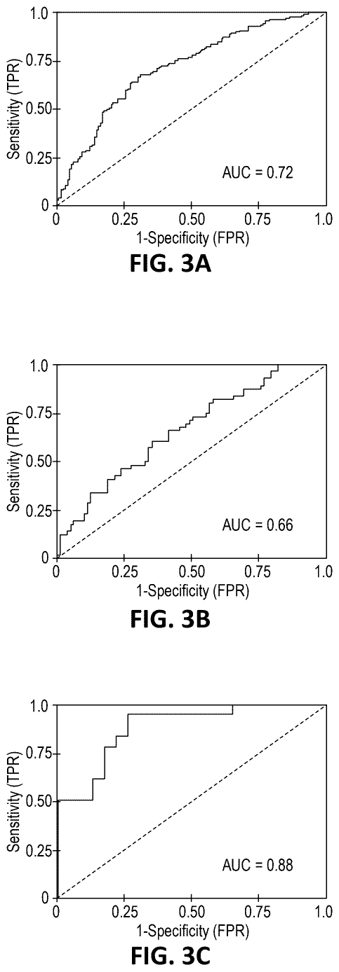

The performance of multivariate logistic regression models of aneurysm rupture (all and smaller than 7 mm) and instability following the stepwise variable selection based on the AIC are presented in Table 4 and FIGS. 3A-3C. These results indicate that aneurysm instability can be forecasted based on aneurysm flow stagnation with approximately 88% accuracy. In addition, aneurysm rupture can be predicted based on aneurysm flow stagnation with approximately 72% accuracy, although this prediction accuracy was slightly reduced to about 66% for aneurysms smaller than 7 mm. The aneurysm-artery difference and ratio of the mean-residence-time (MRT) was retained in all models, indicating that it is a good predictor of aneurysm instability and rupture.

TABLE 2
Univariate analysis comparing contrast retention metrics between ruptured and unruptured
aneurysms from cross-sectional dataset as well as between stable and unstable aneurysms
from longitudinal data set, where statistically significant differences (p <
0.05) are marked by ‘*’, values are given as mean ± standard deviation,
OR represents odds ratio, and AUC represents the area under the ROC curve.
Aneurysm Rupture

| | | Unruptured | Ruptured | | | |
|---|---|---|---|---|---|---|
| Measure | Variable | (n = 129) | (n = 142) | p-value | OR | AUC |
| TT | [symbol] (TT) | 0.78 ± 0.98 | 1.10 ± 1.05 | 0.001* | 1.3 [1.1, 1.7] | 0.61 |
| | [symbol] (TT) | 1.42 ± 0.56 | 1.62 ± 0.61 | 0.0008* | 1.7 [1.1, 2.7] | 0.62 |
| TP | [symbol] (TP) | 0.22 ± 0.22 | 0.18 ± 0.19 | 0.283 | 0.4 [0.1, 1.3] | 0.54 |
| | [symbol] (TP) | 1.88 ± 3.05 | 1.66 ± 2.86 | 0.298 | 0.9 [0.8, 1.1] | 0.54 |
| TW | [symbol] (TW) | 0.55 ± 0.96 | 0.91 ± 1.02 | 0.0004* | 1.4 [1.1, 1.8] | 0.62 |
| | [symbol] (TW) | 1.62 ± 1,13 | 2.01 ± 1.25 | 0.0002* | 1.3 [1.1, 1.6] | 0.63 |
| SK | [symbol] (SK) | −0.03 ± 0.46 | −0.16 ± 0.74 | 0.0001* | 0.6 [0.3, 1.1] | 0.63 |
| | [symbol] (SK) | 2.26 ± 7.38 | 1.21 ± 2.83 | 0.0001* | 0.9 [0.8, 1.1] | 0.64 |
| MRT | [symbol] (MRT) | 0.33 ± 0.35 | 0.33 ± 0.29 | 0.190 | 1.0 [0.5, 2.1] | 0.55 |
| | [symbol] (MRT) | 1.37 ± 0.40 | 1.40 ± 0.39 | 0.038* | 1.2 [0.6, 2.2] | 0.57 |

Small Aneurysm (<7 mm) Rupture

| | | Unruptured | Ruptured | | | |
|---|---|---|---|---|---|---|
| Measure | Variable | (n = 79) | (n = 56) | p-value | OR | AUC |
| TT | [symbol] (TT) | 0.37 ± 0.63 | 0.57 ± 0.75 | 0.039* | 1.5 [0.9, 2.6] | 0.60 |
| | [symbol] (TT) | 1.20 ± 0.36 | 1.33 ± 0.46 | 0.029* | 2.1 [0.9, 5.6] | 0.61 |
| TP | [symbol] (TP) | 0.19 ± 0.24 | 0.16 ± 0.13 | 0.773 | 0.4 [0.05, 2.1] | 0.48 |
| | [symbol] (TP) | 1.90 ± 3.19 | 1.51 ± 2.48 | 0.749 | 0.9 [0.8, 1.1] | 0.48 |
| TW | [symbol] (TW) | 0.16 ± 0.63 | 0.41 ± 0.74 | 0.035* | 1.7 [1.1, 3.0] | 0.61 |
| | [symbol] (TW) | 1.23 ± 0.78 | 1.50 ± 0.96 | 0.017* | 1.4 [0.9, 2.3] | 0.62 |
| SK | [symbol] (SK) | 0.08 ± 0.42 | −0.06 ± 0.37 | 0.019* | 0.3 [0.1, 0.9] | 0.62 |
| | [symbol] (SK) | 2.81 ± 8.93 | 1.38 ± 3.14 | 0.012* | 0.9 [0.8, 1.0] | 0.63 |
| MRT | [symbol] (MRT) | 0.20 ± 0.19 | 0.21 ± 0.17 | 0.686 | 1.2 [0.2, 7.9] | 0.52 |
| | [symbol] (MRT) | 1.22 ± 0.19 | 1.26 ± 0.21 | 0.428 | 2.1 [0.4, 12.4] | 0.54 |

Aneurysm Instability

| | | Stable | Growing | | | |
|---|---|---|---|---|---|---|
| Measure | Variable | (n = 23) | (n = 18) | p-value | OR | AUC |
| TT | [symbol] (TT) | 0.42 ± 1.04 | 1.42 ± 1.25 | 0.029* | 2.55 [1.3, 6.2] | 0.70 |
| | [symbol] (TT) | 1.25 ± 0.39 | 1.81 ± 0.72 | 0.015* | 6.88 [1.9, 41.5] | 0.72 |
| TP | [symbol] (TP) | 0.13 ± 0.48 | 0.22 ± 0.24 | 0.949 | 2.1 [0.4, 30.8] | 0.51 |
| | [symbol] (TP) | 1.95 ± 3.77 | 2.13 ± 2.79 | 0.949 | 1.0 [0.8, 1.2] | 0.49 |
| TW | [symbol] (TW) | 0.28 ± 0.73 | 1.18 ± 1.36 | 0.081 | 2.2 [1.2, 5.0] | 0.66 |
| | [symbol] (TW) | 1.27 ± 0.55 | 2.39 ± 1.60 | 0.034* | 2.7 [1.3, 7.2] | 0.69 |
| SK | [symbol] (SK) | −0.06 ± 0.57 | −0.21 ± 0.67 | 0.086 | 0.6 [0.2, 1.8] | 0.65 |
| | [symbol] (SK) | 2.55 ± 8.06 | 2.81 ± 7.00 | 0.068 | 1.0 [0.9, 1.1] | 0.33 |
| MRT | [symbol] (MRT) | 0.10 ± 0.82 | 0.47 ± 0.32 | 0.011* | 10.1 [1.3, 174.2] | 0.73 |
| | [symbol] (MRT) | 1.22 ± 0.39 | 1.60 ± 0.44 | 0.002* | 13.8 [2.1, 211.4] | 0.77 |

TABLE 3
Contingency table analysis comparing flow stagnation categories obtained
by thresholding different contrast retention metrics and aneurysm rupture
and instability, where statistically significant differences (p < 0.05) are
marked by ‘*’, marginally significant differences (p < 0.06) are marked
with ‘+’, and OR represents odds ratio.
Stagnation vs Rupture

| Variable | 2 × 2 Table | p-value | OR |
|---|---|---|---|
| [symbol] (TT) | $[ \begin{matrix}stag \backslash rupt & U & R \\ N & 86 & 71 \\ Y & 43 & 71\end{matrix} ]$ | 0.006* | 1.99 [1.18, 3.37] |
| [symbol] (TW) | $[ \begin{matrix}stag \backslash rupt & U & R \\ N & 89 & 78 \\ Y & 40 & 64\end{matrix} ]$ | 0.018* | 1.82 [1.07, 3.10] |

Stagnation vs Instability

| Variable | 2 × 2 Table | p-value | OR |
|---|---|---|---|
| [symbol] (TT) | $[ \begin{matrix}stag \backslash grow & S & G \\ N & 17 & 7 \\ Y & 7 & 11\end{matrix} ]$ | 0.059+ | 3.68 [0.88, 16.9] |
| [symbol] (TW) | $[ \begin{matrix}stag \backslash grow & S & G \\ N & 18 & 9 \\ Y & 6 & 10\end{matrix} ]$ | 0.058+ | 3.62 [0.84, 17.1] |

TABLE 4
Multivariate models of aneurysm rupture and instability,
where CV represents cross-validation and AUC represents
the area under the ROC curve.

| | | CV | |
|---|---|---|---|
| Study | Variables Retained | Error | AUC |
| IA rupture | [symbol] (MRT) + [symbol] (MRT) + [symbol] (TW) + [symbol] (TW) | 22.8% | 0.72 |
| Small IA | [symbol] (MRT) + [symbol] (MRT) + [symbol] (TP) + [symbol] (SK) | 24.6% | 0.66 |
| rupture | | | |
| IA instability | [symbol] (MRT) + [symbol] (MRT) | 18.5% | 0.88 |

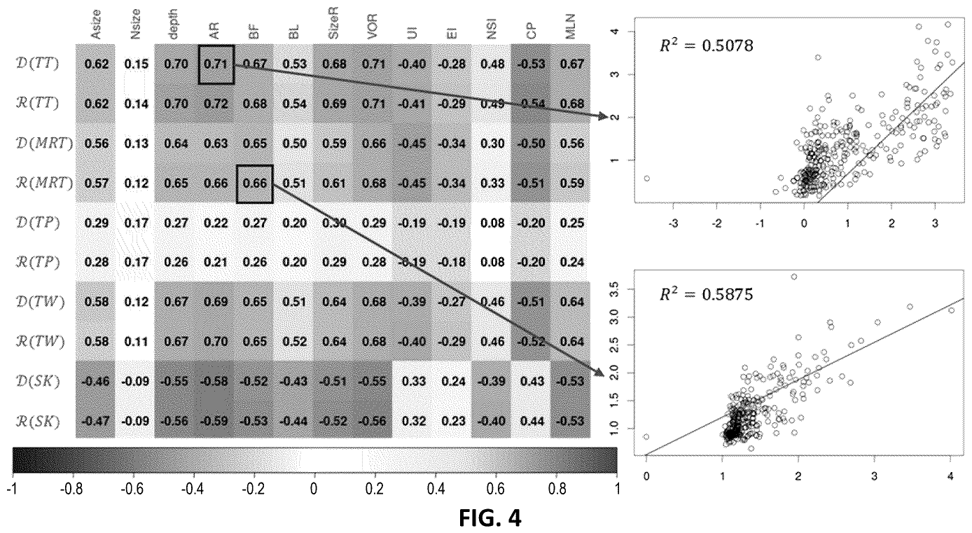

In order to investigate what aneurysm geometries favor flow stagnation, correlations between each of the contrast retention metrics and several aneurysm geometric parameters were investigated using all the data from the cross-sectional dataset. For this purpose, the Spearman correlation coefficient, indicative of a monotonic association between variable ranks, were computed for each variable pair. The results presented in FIG. 4 show that aneurysm aspect ratio (AR), volume-to-ostium ratio (VOR), and bottleneck factor (BF) had the strongest associations with contrast retention variables (e.g., correlations ≥0.70). Additionally, aneurysm depth and size ratio also had some influence on contrast retention but with smaller correlation coefficients. Additionally, the largest linear regression correlation coefficients (R²) were between 0.5 and 0.6 and corresponded to AR and BF (see the right panels of FIG. 4), while aneurysm size metrics (Asize, SizeR) had poorer linear correlations with R²<0.3.

The results of this study suggest that PCOM aneurysm growth and/or development of symptoms related to cranial nerve palsy are associated with or favored by hemodynamic environments characterized by slow and stagnant flows. Furthermore, such flows environments can be recognized by the retention of contrast in angiographic images from CFD-based virtual angiograms (or from real X-ray DSA sequences). Thus, contrast retention can be considered a marker for aneurysm instability (growth and/or symptoms development), which is a recognized risk factor for rupture.

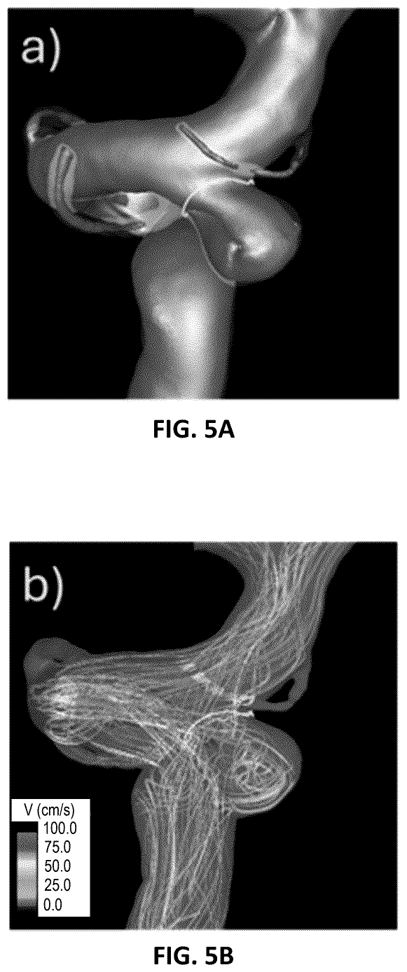

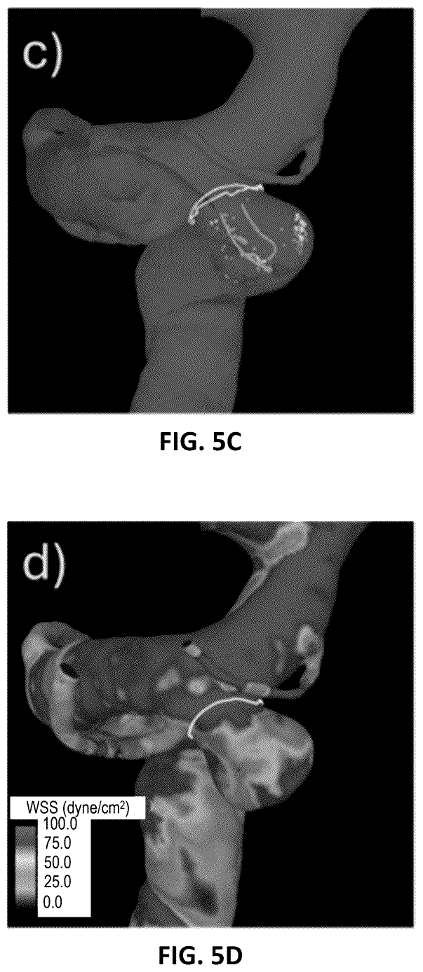

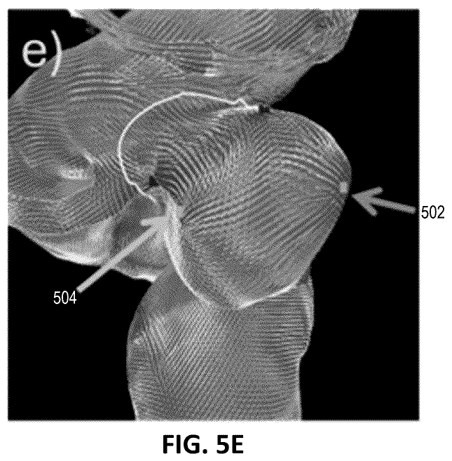

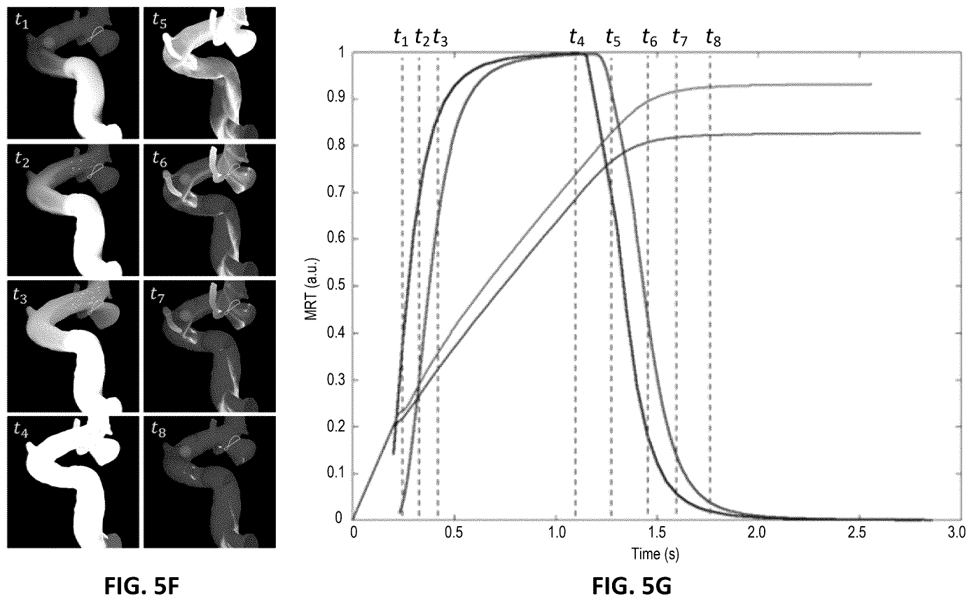

An example of a stable aneurysm with no flow stagnation (e.g., no contrast retention) and analysis thereof is shown in FIGS. 5A-5G. This stable aneurysm shows a fairly strong inflow stream (FIG. 5A) that impacts the aneurysm dome creating a region of elevated wall shear stress with a divergence critical point (node source), and a vortex that produces a swirling critical point (focus) on the aneurysm body (FIG. 5E). The corresponding virtual angiogram (FIG. 5F) and time density curves (FIG. 5G) show that the aneurysm fills slightly after the proximal parent artery region and washes out in roughly the same amount of time, thus not exhibiting any significant contrast retention.

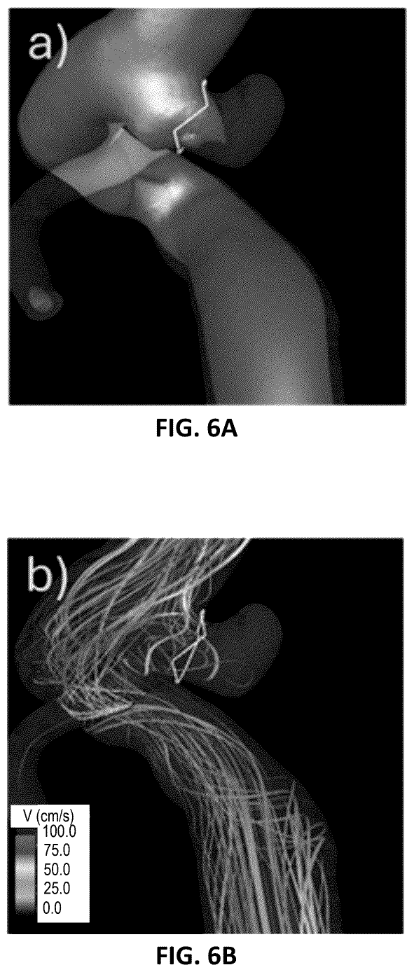

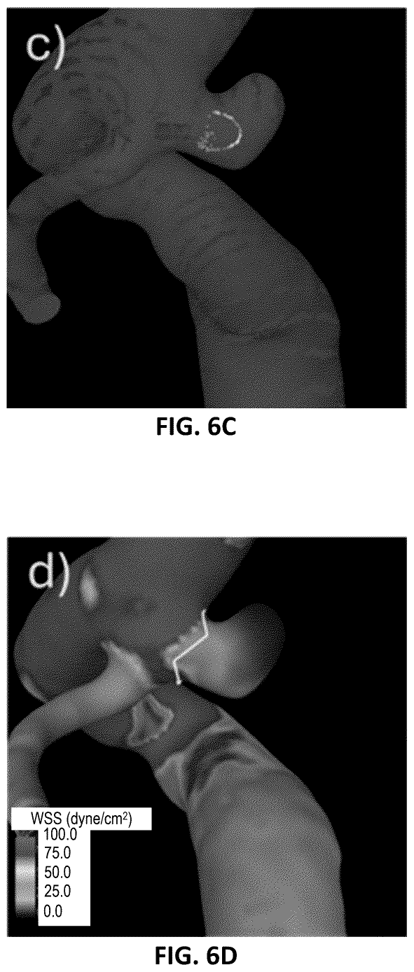

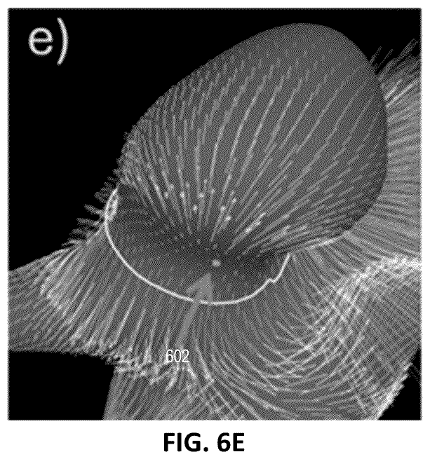

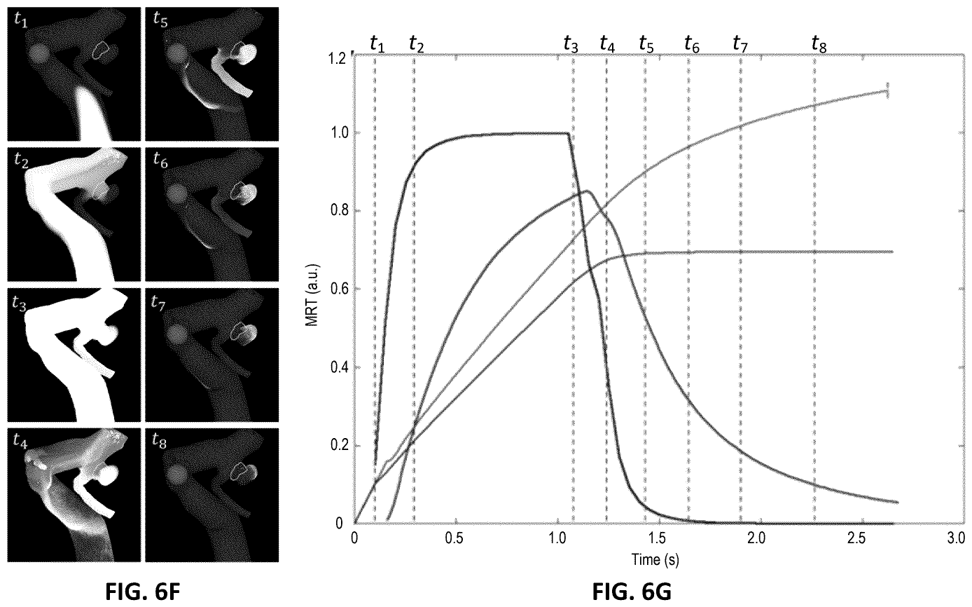

An example of an unstable (growing) aneurysm with significant contrast retention and analysis thereof is shown in FIGS. 6A-6G. This unstable aneurysm shows an inflow stream (FIG. 6A) impacting the distal part of the neck and entering the aneurysm with much slower velocity, thus creating a vortex structure and a low-velocity, low-wall-shear-stress (WSS) zone toward the dome. A node critical point can be observed in the outflow zone near the neck, as shown in FIG. 6E. Well after the proximal parent artery is washed out, contrast retention can still be observed in the aneurysm, especially toward the distal part of the dome, as reflected in the corresponding virtual angiogram (FIG. 6F) and time density curves (FIG. 6G). It is interesting to note that this aneurysm exhibited contrast retention even though it was quite small (e.g., 3 mm).

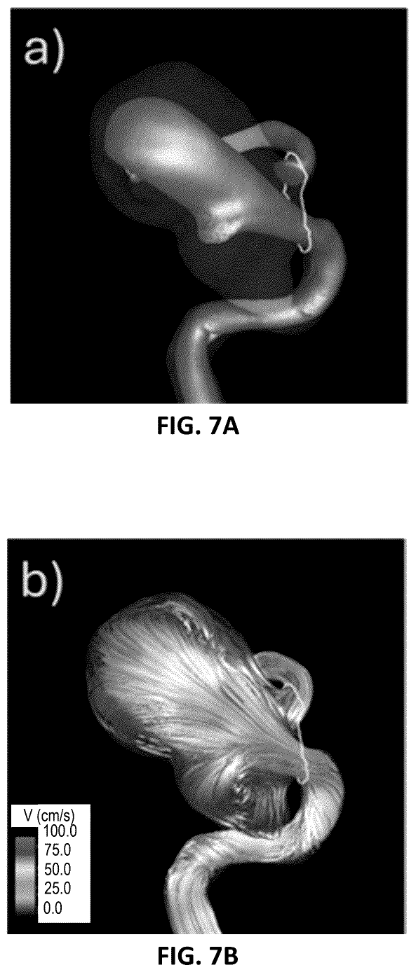

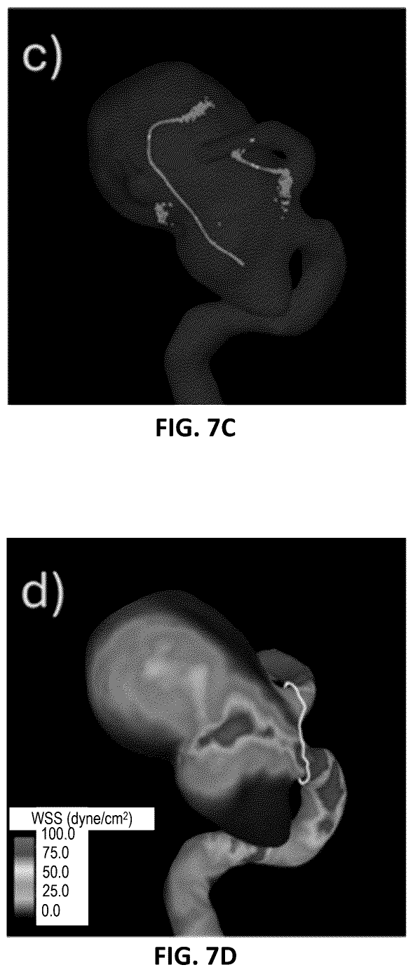

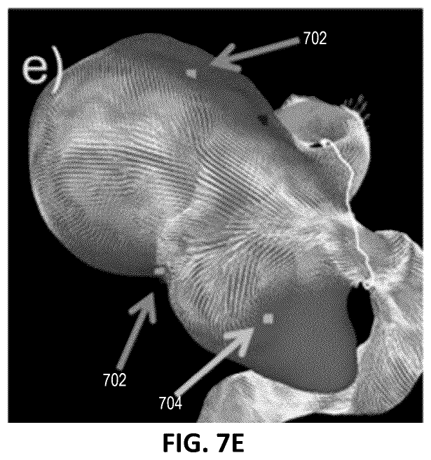

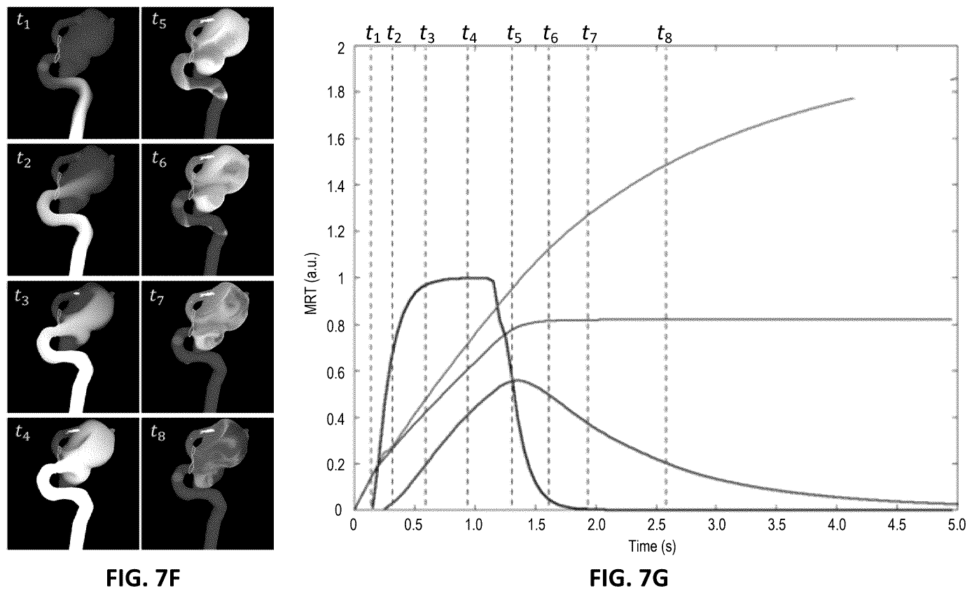

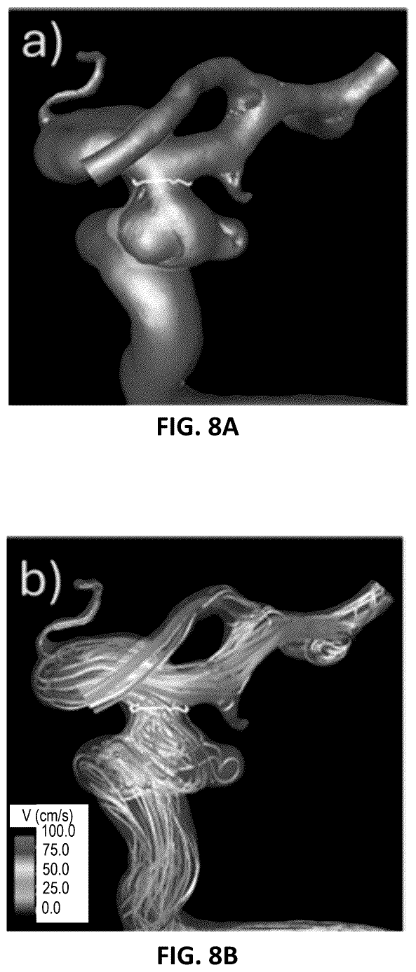

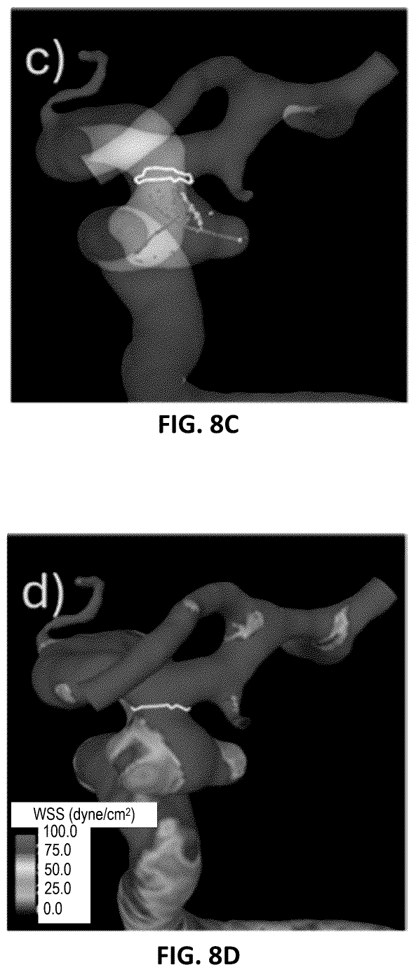

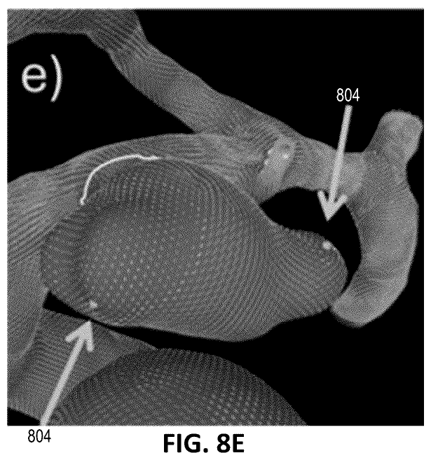

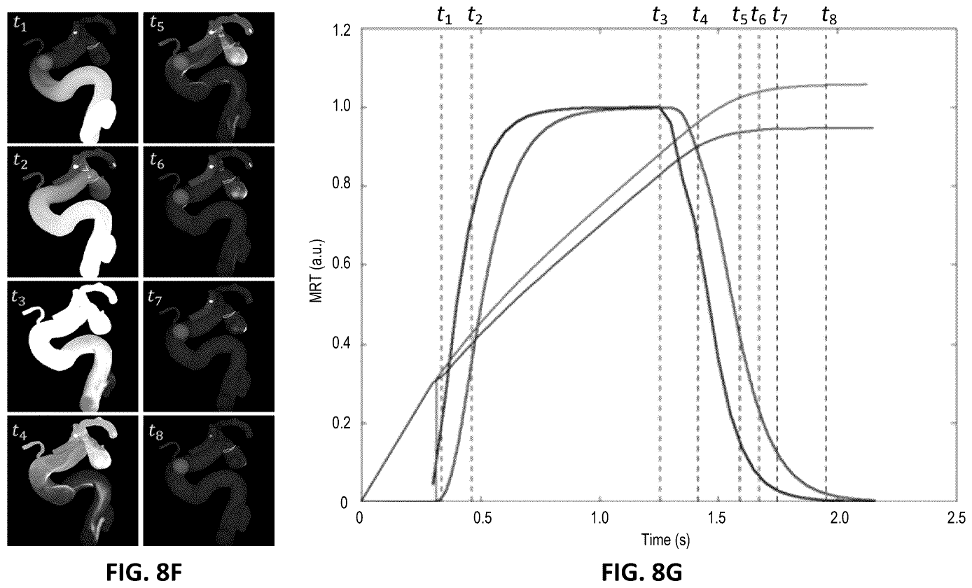

This study also found associations between contrast retention and aneurysm rupture. However, it showed that rupture can also occur under high flow conditions without flow stagnation and contrast retention, especially if considering only smaller aneurysms. Examples of ruptured aneurysms with and without contrast retention, and corresponding analyses thereof, are shown in FIGS. 7A-7G and FIGS. 8A-8G, respectively. In both cases, there was a strong inflow jet that impacted the wall creating a region of elevated WSS and vortices ending at the wall with swirling critical points. In the first case (FIGS. 7A-7G), there was substantial contrast retention, especially at the wall opposite to the flow impingement that was subjected to low WSS. In contrast, in the second case (FIGS. 8A-8G), the flow stream was strong throughout the aneurysm and able to quickly wash out the contrast without significant retention.

These findings and observations suggest at least two underlying mechanisms responsible for aneurysm wall degeneration, weakening, and subsequent rupture-one related to high flow effects and another related to low and stagnant flow effects. Furthermore, these two kinds of local hemodynamic conditions can co-exist simultaneously in a single aneurysm, resulting in a heterogeneous wall and arguably a more vulnerable wall structure.

Although flow stagnation was weakly related to size, it was found to depend on aneurysm shape, in particular, to geometric parameters related to its elongation (e.g., aspect ratio) or inflation (e.g., bottleneck factor or volume-to-ostium ratio) with respect to its neck. In other words, larger and/or wider aneurysms with smaller necks are likely to produce stagnant flows that favor further growth (and perhaps also wall thickening) and increased potential for future rupture. Although stable aneurysms had less flow stagnation in general, the absence of contrast retention should not be considered an indication of low rupture risk, since many ruptured aneurysms did not exhibit significant contrast retention. In the absence of contrast retention, other flow conditions and rupture risk factors (e.g., strong impingements, vortex flows, complex and unstable flow patterns, etc.) should be further analyzed to assess the likelihood of aneurysm rupture without stagnation.

### CONCLUSION

Any of the features illustrated or described herein, for example, with respect to FIGS. 1A-8G, can be combined with any other feature illustrated or described herein, for example, with respect to FIGS. 1A-8G to provide systems, devices, modules, methods, and embodiments not otherwise illustrated or specifically described herein. All features described herein are independent of one another and, except where structurally impossible, can be used in combination with any other feature described herein. In view of the many possible embodiments to which the principles of the disclosed technology may be applied, it should be recognized that the illustrated embodiments are only examples and should not be taken as limiting the scope of the disclosed technology. Rather, the scope is defined by the following claims. Applicant therefore claims all that comes within the scope and spirit of these claims.

1. A method comprising:
performing a virtual angiogram by simulating transport of contrast agent through a model of a portion of a vasculature of a patient based on a patient-specific pulsatile flow velocity field of said vasculature portion, the vasculature portion including an aneurysm that has not ruptured;
determining at least one contrast retention metric for the aneurysm based on the virtual angiogram, the at least one contrast retention metric being indicative of flow stagnation in the aneurysm; and
identifying the aneurysm as unstable based at least in part on the at least one contrast retention metric.

2. The method of claim 1, further comprising:
physically administering an endovascular, surgical, or radiosurgical treatment to stabilize, occlude, or obliterate the aneurysm prior to rupture, in response to the identifying as unstable.

3. The method of claim 1, further comprising, prior to the performing the virtual angiogram:
obtaining the patient-specific pulsatile flow velocity field via 4D Flow Magnetic Resonance Imaging (MRI) of the patient.

4. The method of claim 1, further comprising, prior to the performing the virtual angiogram:
constructing a model of the vasculature portion based at least in part on 3D medical data of the patient; and
performing computational fluid dynamic (CFD) simulations of the constructed model for pulsatile flow conditions to obtain the patient-specific pulsatile flow velocity field.

5. The method of claim 4, further comprising, prior to the constructing the model of the vasculature portion:
obtaining the 3D medical data via a computed tomography angiogram (CTA) or magnetic resonance angiogram (MRA) utilizing intravenous contrast administration.

6. The method of claim 1, wherein the simulating transport of the contrast agent comprises solving an advection transport equation over the patient-specific pulsatile flow velocity field.

7. The method of claim 1, wherein the determining at least one contrast retention metric comprises:
identifying a first region in the model corresponding to the aneurysm and a second region in the model corresponding to a parent artery proximal to the aneurysm;
calculating time density curves for contrast agent concentration in each of the first and second regions; and
calculating the at least one contrast retention metric based at least in part on the calculated time density curves.

8. The method of claim 7, wherein:
the calculating the at least one contrast retention metric comprises determining at least one regional metric for each of the first and second regions based on the calculated time density curves,
the at least one contrast retention metric is calculated as (i) a difference between the regional metric for the first region and a corresponding regional metric for the second region, or (ii) a ratio of the regional metric for the first region and the corresponding regional metric for the second region, and
the aneurysm is identified as unstable responsive to the at least contrast retention metric being greater than a predetermined threshold.

9. The method of claim 8, wherein the at least one contrast retention metric comprises a difference or ratio of regional metrics for the first and second regions, and the regional metrics are transit time or mean residence time.

10. The method of claim 1, wherein the aneurysm is a cerebral aneurysm having a maximum dimension of less than or equal to 7 millimeters.

11. A method comprising:
performing a virtual angiogram by simulating transport of contrast agent through a model of a portion of a vasculature of a patient based on a patient-specific pulsatile flow velocity field of said vasculature portion, the vasculature portion including a plurality of aneurysms, at least one of the aneurysms being ruptured;
determining at least one contrast retention metric for each of the plurality of aneurysms based on the virtual angiogram, each contrast retention metric being indicative of flow stagnation in the respective aneurysm;
identifying at least one of the plurality of aneurysms as ruptured based at least in part on the contrast retention metrics; and
physically administering an endovascular, surgical, or radiosurgical treatment to stabilize, occlude, or obliterate the at least one aneurysm identified as ruptured.

12. The method of claim 11, wherein the identifying comprises:
selecting one of the aneurysms from the plurality that has an extremum for the determined contrast retention metrics, and identifying the selected one as ruptured; or
identifying each aneurysm that has its respective contrast retention metric greater than a predetermined threshold as being ruptured.

13. The method of claim 11, further comprising:
displaying (i) an image of the vasculature portion showing the plurality of aneurysms and (ii) respective contrast retention metrics for the aneurysms and/or indication of the at least one aneurysm identified as ruptured.

14. The method of claim 11, further comprising, prior to the performing the virtual angiogram:
obtaining the patient-specific pulsatile flow velocity field via 4D Flow Magnetic Resonance Imaging (MRI) of the patient; or
constructing a model of the vasculature portion based at least in part on 3D medical data of the patient, and performing computational fluid dynamic (CFD) simulations of the constructed model for pulsatile flow conditions to obtain the patient-specific pulsatile flow velocity field.

15. The method of claim 14, further comprising, prior to the constructing the model of the vasculature portion:
obtaining the 3D medical data via a computed tomography angiogram (CTA) or magnetic resonance angiogram (MRA) utilizing intravenous contrast administration,
wherein the virtual angiogram mathematically simulates local contrast transport in the vasculature portion of the patient without requiring a physical intra-arterial catheterization of the patient.

16. The method of claim 11, wherein the determining at least one contrast retention metric comprises, for each of the plurality of aneurysms:
identifying a first region in the model corresponding to the aneurysm and a second region in the model corresponding to a parent artery proximal to the aneurysm;
calculating time density curves for contrast agent concentration in each of the first and second regions;
determining at least one regional metric for each of the first and second regions based on the calculated time density curves; and
calculating the at least one contrast retention metric as (i) a difference between the regional metric for the first region and a corresponding regional metric for the second region, or (ii) a ratio of the regional metric for the first region and the corresponding regional metric for the second region,
wherein the at least one of the plurality of aneurysms is identified as ruptured responsive to the corresponding contrast retention metric being greater than a predetermined threshold or being an extremum with respect to the contrast retention metrics calculated for the plurality of aneurysms.

17. An aneurysm assessment system comprising:
one or more processors; and
one or more non-transitory media storing computer-readable instructions that, when executed by the one or more processors, cause the one or more processors to:
perform a virtual angiogram by simulating transport of contrast agent through a model of a portion of a vasculature of a patient based on a patient-specific pulsatile flow velocity field of said vasculature portion, the vasculature portion including at least one aneurysm;
determine at least one contrast retention metric for the at least one aneurysm based on the virtual angiogram, the at least one contrast retention metric being indicative of flow stagnation in the respective aneurysm; and
identify the at least one aneurysm as stable, unstable, or ruptured based at least in part on the at least one contrast retention metric.

18. The aneurysm assessment system of claim 17, wherein the one or more non-transitory media store additional computer-readable instructions that, when executed by the one or more processors, further cause the one or more processors to:
construct a model of the vasculature portion based at least in part on 3D medical data of the patient; and
perform computational fluid dynamic (CFD) simulations of the constructed model for pulsatile flow conditions to obtain the patient-specific pulsatile flow velocity field.

19. The aneurysm assessment system of claim 17, wherein the one or more non-transitory media store computer-readable instructions that, when executed by the one or more processors, cause the one or more processors to determine the at least one contrast retention metric by:
identifying a first region in the model corresponding to the at least one aneurysm and a second region in the model corresponding to a parent artery proximal to the aneurysm;
calculating time density curves for contrast agent concentration in each of the first and second regions;
determine at least one regional metric for each of the first and second regions based on the calculated time density curves; and
calculating the at least one contrast retention metric as (i) a difference between the regional metric for the first region and a corresponding regional metric for the second region, or (ii) a ratio of the regional metric for the first region and the corresponding regional metric for the second region.

20. The aneurysm assessment system of claim 17, wherein the one or more non-transitory media store additional computer-readable instructions that, when executed by the one or more processors, further cause the one or more processors to:
output to a display overlaying the at least one determined contrast retention metric onto a visualization of the vascular portion to guide a physical treatment intervention.
